SEQUENCE LISTING (64 sequences)

SEQ ID NO: 1 - DNA, 18 bp, Artificial Sequence
  Description of Artificial Sequence Synthetic oligonucleotide
tgggtagggcgggtggga

SEQ ID NO: 2 - RNA, 43 nt, Artificial Sequence
  Description of Artificial Sequence Synthetic oligonucleotide
gaccaccccaaaaaugaaggggacuuggguagggcggguggga

SEQ ID NO: 3 - RNA, 50 nt, Artificial Sequence
  Description of Artificial Sequence Synthetic oligonucleotide
gaccaccccaaaaaugaaggggacuaaaacuuucgcugauuuuggggucc

SEQ ID NO: 4 - DNA, 51 bp, Artificial Sequence
  Description of Artificial Sequence Synthetic oligonucleotide
  Description of Combined DNA/RNA Molecule Synthetic oligonucleotide
uuuuuucccacccgcccuacccauuuuuuuuuutgggtagggcgggtggga

SEQ ID NO: 5 - RNA, 61 nt, Artificial Sequence
  Description of Artificial Sequence Synthetic oligonucleotide
uuuuuggaccccaaaaucagcgaaaguuuuuuuuuuaaaacuuucgcugauuuuggggucc

SEQ ID NO: 6 - DNA, 15 bp, Artificial Sequence
  Description of Artificial Sequence Synthetic oligonucleotide
cacccgctctaccca

SEQ ID NO: 7 - DNA, 43 bp, Artificial Sequence
  Description of Artificial Sequence Synthetic oligonucleotide
cacccgctctacccatttttttttttgggtagggcgggtggga

SEQ ID NO: 8 - DNA, 15 bp, Artificial Sequence
  Description of Artificial Sequence Synthetic oligonucleotide
tgggtagggcgggtg

SEQ ID NO: 9 - DNA, 15 bp, Artificial Sequence
  Description of Artificial Sequence Synthetic oligonucleotide
cactcgctctaccca

SEQ ID NO: 10 - DNA, 24 bp, Artificial Sequence
  Description of Artificial Sequence Synthetic oligonucleotide
ttattacgcttaccatccatattt

SEQ ID NO: 11 - DNA, 83 bp, Artificial Sequence
  Description of Artificial Sequence Synthetic oligonucleotide
cttacgttctgttacttactgccgagactatcaaatatggatggtaagcgtggtcgatcgaggttcggccatcgccgtcttta

SEQ ID NO: 12 - DNA, 32 bp, Artificial Sequence
  Description of Artificial Sequence Synthetic oligonucleotide
gatagtctcggcagtacgaacctcgatcgacc

SEQ ID NO: 13 - DNA, 32 bp, Artificial Sequence
  Description of Artificial Sequence Synthetic oligonucleotide
agtaacagaacgtaagtaaagacggcgatggc

SEQ ID NO: 14 - DNA, 32 bp, Artificial Sequence
  Description of Artificial Sequence Synthetic oligonucleotide
cttacgttctgttacttactgccgagactatc

SEQ ID NO: 15 - DNA, 51 bp, Artificial Sequence
  Description of Artificial Sequence Synthetic oligonucleotide
agtaacagaacgtaagtgggtagggcgggttgggagatagtctcggcagta

SEQ ID NO: 16 - DNA, 30 bp, Artificial Sequence
  Description of Artificial Sequence Synthetic oligonucleotide
cttacgttctgttacttactgccgagacta

SEQ ID NO: 17 - DNA, 43 bp, Artificial Sequence
  Description of Artificial Sequence Synthetic oligonucleotide
ctcggcagtaattgggtagggcgggttgggatgtaacagaacg

SEQ ID NO: 18 - DNA, 72 bp, Artificial Sequence
  Description of Artificial Sequence Synthetic oligonucleotide
cttacgttctgttacttactgccgagactatcaaatatggatggtaagcgtggtcgatcgaggttcggccat

SEQ ID NO: 19 - DNA, 59 bp, Artificial Sequence
  Description of Artificial Sequence Synthetic oligonucleotide
  misc_feature (59)..(59): may or may not be present
tgggaatggccgaacctcgatcgacagtaagtaacagaacgtaagtgggtagggcgggt

SEQ ID NO: 20 - DNA, 32 bp, Artificial Sequence
  Description of Artificial Sequence Synthetic oligonucleotide
atttgatagtctcggccacgcttaccatccat

SEQ ID NO: 21 - DNA, 64 bp, Artificial Sequence
  Description of Artificial Sequence Synthetic oligonucleotide
cttacgttctgttacttactgccgagactatcaaatatggatggtaagcgtggtcgatcgaggt

SEQ ID NO: 22 - DNA, 51 bp, Artificial Sequence
  Description of Artificial Sequence Synthetic oligonucleotide
  misc_feature (51)..(51): may or may not be present
tgggaacctcgatcgaccacgagtaacagaacgtaagtgggtagggcgggt

SEQ ID NO: 23 - DNA, 32 bp, Artificial Sequence
  Description of Artificial Sequence Synthetic oligonucleotide
gatagtctcggcagtacttaccatccatattt

SEQ ID NO: 24 - DNA, 59 bp, Artificial Sequence
  Description of Artificial Sequence Synthetic oligonucleotide
  misc_feature (24)..(24): may or may not be present
gaacgtaagtgggtagggcgggttgggaatggccgaacctcgatcgacagtaagtaaca

SEQ ID NO: 25 - DNA, 51 bp, Artificial Sequence
  Description of Artificial Sequence Synthetic oligonucleotide
  misc_feature (23)..(23): may or may not be present
aacgtaagtgggtagggcgggttgggaacctcgatcgaccacgagtaacag

SEQ ID NO: 26 - DNA, 38 bp, Artificial Sequence
  Description of Artificial Sequence Synthetic oligonucleotide
tttatttgatagtctcggccacgcttaccatccatttt

SEQ ID NO: 27 - DNA, 38 bp, Artificial Sequence
  Description of Artificial Sequence Synthetic oligonucleotide
tttgatagtctcggcagtacttaccatccatatttttt

SEQ ID NO: 28 - DNA, 58 bp, Artificial Sequence
  Description of Artificial Sequence Synthetic oligonucleotide
tgggaatggccgaacctcgatcgacagtaagtaacagaacgtaagtgggtagggcggg

SEQ ID NO: 29 - DNA, 50 bp, Artificial Sequence
  Description of Artificial Sequence Synthetic oligonucleotide
tgggaacctcgatcgaccacgagtaacagaacgtaagtgggtagggcggg

SEQ ID NO: 30 - DNA, 58 bp, Artificial Sequence
  Description of Artificial Sequence Synthetic oligonucleotide
gaacgtaagtgggtagggcgggtgggaatggccgaacctcgatcgacagtaagtaaca

SEQ ID NO: 31 - DNA, 50 bp, Artificial Sequence
  Description of Artificial Sequence Synthetic oligonucleotide
aacgtaagtgggtagggcgggtgggaacctcgatcgaccacgagtaacag

SEQ ID NO: 32 - DNA, 32 bp, Artificial Sequence
  Description of Artificial Sequence Synthetic oligonucleotide
acctcgatcgaccacgcttaccatccatattt

SEQ ID NO: 33 - DNA, 96 bp, Artificial Sequence
  Description of Artificial Sequence Synthetic oligonucleotide
cttacgttctgttacttactgccgagactatcaaatatggatggtaagcgtggtcgatcgaggttcggccatcgccgtctttaccaaattgtaatt

SEQ ID NO: 34 - DNA, 32 bp, Artificial Sequence
  Description of Artificial Sequence Synthetic oligonucleotide
gatagtctcggcagtaagacggcgatggccga

SEQ ID NO: 35 - DNA, 50 bp, Artificial Sequence
  Description of Artificial Sequence Synthetic oligonucleotide
aacgtaagtgggtagggcgggtgggaaattacaatttggtaaagtaacag

SEQ ID NO: 36 - DNA, 35 bp, Artificial Sequence
  Description of Artificial Sequence Synthetic oligonucleotide
cttacgttctgttacttactgccgagactatcaaa

SEQ ID NO: 37 - DNA, 58 bp, Artificial Sequence
  Description of Artificial Sequence Synthetic oligonucleotide
  Description of Combined DNA/RNA Molecule Synthetic oligonucleotide
taagtaacagaacgtaaguuuuutgggtagggcgggtgggatttgatagtctcggcag

SEQ ID NO: 38 - DNA, 59 bp, Artificial Sequence
  Description of Artificial Sequence Synthetic oligonucleotide
  Description of Combined DNA/RNA Molecule Synthetic oligonucleotide
gccgaacctcgatcgaccauuuuutgggtagggcgggtgggaaagtaacagaacgtaag

SEQ ID NO: 39 - DNA, 69 bp, Artificial Sequence
  Description of Artificial Sequence Synthetic oligonucleotide
cttacgttctgttacttactgccgagactatcaaatatggatggtaagcgtggtcgatcgaggttcggc

SEQ ID NO: 40 - DNA, 33 bp, Artificial Sequence
  Description of Artificial Sequence Synthetic oligonucleotide
cgcttaccatccatatttgatagtctcggcagt

SEQ ID NO: 41 - DNA, 51 bp, Artificial Sequence; identical to SEQ ID NO: 25
  Description of Artificial Sequence Synthetic oligonucleotide

SEQ ID NO: 42 - DNA, 19 bp, Artificial Sequence
  Description of Artificial Sequence Synthetic oligonucleotide
tggtcgatcgaggttcggc

SEQ ID NO: 43 - DNA, 38 bp, Artificial Sequence
  Description of Artificial Sequence Synthetic oligonucleotide
catacaaccatcccacccgccctacccaccactcacac

SEQ ID NO: 44 - DNA, 38 bp, Artificial Sequence
  Description of Artificial Sequence Synthetic oligonucleotide
gtgtgagtggtgggtagggcgggtgggatggttgtatg

SEQ ID NO: 45 - DNA, 45 bp, Artificial Sequence
  Description of Artificial Sequence Synthetic oligonucleotide
catacaaccatcccaccctttttttgccctacccaccactcacac

SEQ ID NO: 46 - DNA, 45 bp, Artificial Sequence
  Description of Artificial Sequence Synthetic oligonucleotide
  Description of Combined DNA/RNA Molecule Synthetic oligonucleotide
catacaaccatcccacccuuuuuuugccctacccaccactcacac

SEQ ID NO: 47 - DNA, 52 bp, Artificial Sequence
  Description of Artificial Sequence Synthetic oligonucleotide
catacaaccatccctttttttacccgcccttttttttacccaccactcacac

SEQ ID NO: 48 - DNA, 52 bp, Artificial Sequence
  Description of Artificial Sequence Synthetic oligonucleotide
  Description of Combined DNA/RNA Molecule Synthetic oligonucleotide
catacaaccatcccuuuuuuuacccgccctuuuuuuuacccaccactcacac

SEQ ID NO: 49 - DNA, 57 bp, Artificial Sequence
  Description of Artificial Sequence Synthetic oligonucleotide
catacaacctttttttatcccacccgtttttccctacccatttttttccactcacac

SEQ ID NO: 50 - DNA, 57 bp, Artificial Sequence
  Description of Artificial Sequence Synthetic oligonucleotide
  Description of Combined DNA/RNA Molecule Synthetic oligonucleotide
catacaaccuuuuuuuatcccacccguuuuuccctacccauuuuuuuccactcacac

SEQ ID NO: 51 - DNA, 54 bp, Artificial Sequence
  Description of Artificial Sequence Synthetic oligonucleotide
ctgccgagactatcaaaatcccacccgccctacccacttacgttctgttactta

SEQ ID NO: 52 - DNA, 54 bp, Artificial Sequence
  Description of Artificial Sequence Synthetic oligonucleotide
taagtaacagaacgtaagtgggtagggcgggtgggattttgatagtctcggcag

SEQ ID NO: 53 - DNA, 61 bp, Artificial Sequence
  Description of Artificial Sequence Synthetic oligonucleotide
ctgccgagactatcaaaatcccaccctttttttgccctacccacttacgttctgttactta

SEQ ID NO: 54 - DNA, 61 bp, Artificial Sequence
  Description of Artificial Sequence Synthetic oligonucleotide
  Description of Combined DNA/RNA Molecule Synthetic oligonucleotide
ctgccgagactatcaaaatcccacccuuuuuuugccctacccacttacgttctgttactta

SEQ ID NO: 55 - DNA, 68 bp, Artificial Sequence
  Description of Artificial Sequence Synthetic oligonucleotide
ctgccgagactatcaaaatccctttttttacccgcccttttttttacccacttacgttctgttactta

SEQ ID NO: 56 - DNA, 68 bp, Artificial Sequence
  Description of Artificial Sequence Synthetic oligonucleotide
  Description of Combined DNA/RNA Molecule Synthetic oligonucleotide
ctgccgagactatcaaaatcccuuuuuuuacccgccctuuuuuuuacccacttacgttctgttactta

SEQ ID NO: 57 - DNA, 73 bp, Artificial Sequence
  Description of Artificial Sequence Synthetic oligonucleotide
ctgccgagactatcaaatttttttatcccaccctttttgccctacccatttttttcttacgttctgttactta

SEQ ID NO: 58 - DNA, 73 bp, Artificial Sequence
  Description of Artificial Sequence Synthetic oligonucleotide
  Description of Combined DNA/RNA Molecule Synthetic oligonucleotide
ctgccgagactatcaaauuuuuuuatcccacccuuuuugccctacccauuuuuuucttacgttctgttactta

SEQ ID NO: 59 - RNA, 18 nt, Artificial Sequence
  Description of Artificial Sequence Synthetic oligonucleotide
uggguagggcggguggga

SEQ ID NO: 60 - RNA, 18 nt, Artificial Sequence
  Description of Artificial Sequence Synthetic oligonucleotide
ucccacccgcccuaccca

SEQ ID NO: 61 - DNA, 19 bp, Artificial Sequence
  Description of Artificial Sequence Synthetic oligonucleotide
tgggtagggcgggttggga

SEQ ID NO: 62 - DNA, 59 bp, Artificial Sequence; identical to SEQ ID NO: 19
  Description of Artificial Sequence Synthetic oligonucleotide

SEQ ID NO: 63 - DNA, 51 bp, Artificial Sequence; identical to SEQ ID NO: 22
  Description of Artificial Sequence Synthetic oligonucleotide

SEQ ID NO: 64 - DNA, 59 bp, Artificial Sequence; identical to SEQ ID NO: 24
  Description of Artificial Sequence Synthetic oligonucleotide

(19) United States
(12) Patent Application Publication
(10) Pub. No.: US 2024/0018607 A1
(43) Pub. Date: Jan. 18, 2024

(54) MOLECULAR BEACONS

(71) Applicant: LogicInk Corporation, San Francisco, CA (US)

(72) Inventors: Carlos Edel OLGUIN ALVAREZ, San Francisco, CA (US); Hendrik DIETZ, Haar (DE); Steven WROBEL, San Francisco, CA (US)

(21) Appl. No.: 18/252,869

(22) PCT Filed: Nov. 12, 2021
(86) PCT No.: PCT/US2021/059212

§ 371 Date: May 12, 2023

Related U.S. Application Data
(63) Continuation-in-part of application No. 17/219,527, filed on Mar. 31, 2021.
(60) Provisional application No. 63/215,834, filed on Jun. 28, 2021.
(60) Provisional application No. 63/214,428, filed on Jun. 24, 2021.
(60) Provisional application No. 63/181,891, filed on Apr. 29, 2021.
(60) Provisional application No. 63/002,960, filed on Mar. 31, 2020.
(60) Provisional application No. 63/149,147, filed on Feb. 12, 2021.
(60) Provisional application No. 63/125,649, filed on Dec. 15, 2020.
(60) Provisional application No. 63/113,672, filed on Nov. 13, 2020.

(51) Int. Cl. C12Q 1/6897; C12Q 1/44

(52) U.S. Cl. CPC C12Q 1/6897; C12Q 1/44

(57) ABSTRACT

Systems comprising a translation module, an amplification module comprising one or more sensing modalities, and a detection module configured to accept the amplifier output and methods of using the same are provided. The systems or methods can be capable of detecting trace amounts of nucleic acid molecules with high sensitivity and specificity in a device-free system with naked-eye readable output. The systems can comprise three layers incorporated into a lateral flow assay and/or a paper strip assay, in such systems, the first layer can comprise the translation module, the second layer can comprise the amplification module, and the third layer can comprise the detection module.

### CROSS-REFERENCE TO RELATED APPLICATIONS

This application is a continuation-in-part of currently pending U.S. patent application Ser. No. 17/219,527 entitled “System For Detection of A Target Analyte Via Self-Testing, Object Surfaces, And The Environment,” filed Mar. 31, 2021, which claims priority to U.S. Provisional Application No. 63/002,960; and claims priority under 35 U.S.C. § 119(e) to U.S. Provisional Application No. 63/113,672 entitled “DNA Origami-Based Logic Control of A Peroxidase,” filed Nov. 13, 2020; U.S. Provisional Application No. 63/125,649 entitled “Logic Control of A Peroxidase,” filed Dec. 15, 2020; U.S. Provisional Application No. 63/149,147 entitled “Colorimetric Molecular Beacon,” filed Feb. 12, 2021; U.S. Provisional Application No. 63/181,891 entitled “Genetic Signal,” filed Apr. 29, 2021; U.S. Provisional Application No. 63/214,428 entitled “Molecular Beacons,” filed Jun. 24, 2021; and U.S. Provisional Application No. 63/215,834 entitled “Exponential Increase of Indiscriminate Nuclease Activity,” filed Jun. 28, 2021, each of which is incorporated by reference herein in its entirety.

All patents, patent applications, and publications cited herein are hereby incorporated by reference in their entirety in order to more fully describe the state of the art as known to those skilled therein as of the date of the invention described herein.

### SEQUENCE LIST

The instant application contains a Sequence Listing which has been submitted electronically in ASCII format and is hereby incorporated by reference in its entirety. Said ASCII copy, created on Nov. 8, 2021, is named 2212508_00136WO3_SL.txt and is 17,536 bytes in size.

### BACKGROUND

### Field of the Invention

This application generally relates to systems and methods to detect nucleic acid and/or small molecule targets and/or to provide a readily understandable readout indicating the presence or absence of the target(s).

### Description of Related Art

There is a need for simple and readily understandable, low-cost, non-invasive, semi-continuous use, accumulated and/or real-time multi-sensing of markers from a broad set of sources including human bodies, animals, object surfaces, environments without requiring the use of additional electronic devices.

A person is exposed to a range of environments on a daily basis. The conditions of these environments and the length of exposure to these conditions may impact a person's mental and/or physical state. Several of these conditions may go undetected. Further, their impact on a person exposed to these conditions are not immediately apparent. For example, the microbiome present on a person's skin may be indicative of the individual's health and is not immediately apparent.

Human bodies are continuously exposed to microbial cells and their byproducts which can include toxic metabolites. Circulation of toxic metabolites may contribute to the onset of cancer. In addition, microbes may migrate throughout the human body and become associated with tumor development. Further, the presence or absence of specific microflora in a microbiome has been found to be associated with various health conditions including cancer, chronic inflammation, hydration levels, skin hydration levels, immune system disfunction, atopic dermatitis, psoriasis, acne vulgaris, skin ulcers, and conditions associated with aging. These microbiomes include those from a subject's gut, skin, and other topical areas of the body.

Additionally, a person commonly comes in contact with a myriad of infectious agents including microbial cells such as bacterial cells and virions. An immediately pressing example of an environmental condition that is not immediately apparent is the presence of viral components, such as those of the novel corona virus (e.g., SARS-CoV-2). Thus, it would be beneficial to identify and analyze the presence of infections agents such as viral components to determine exposure to such components.

Thus, it would be beneficial to identify and analyze the microbiome of a subject (or nucleic acids and/or small molecules thereof) and/or indicators of environmental exposures that may be deleterious (e.g., nucleic acids present in the SARS-CoV-2 virion or produced by the human body as a consequence of exposure to SARS-CoV-2) to be used to detect or predict via correlation the occurrence of carcinogenic conditions, inflammation disorders, potential infections, and other health conditions.

Despite the above needs, traditional electronic devices are bulky, battery-powered, expensive, and/or difficult to learn. Further, previous technology commonly relied on not readily available laboratory equipment such as gel electrophoretic equipment. Thus, there is a need for technology that is capable of identifying and analyzing the instant condition or exposure and highly sensitive, specific, low-cost, instrument-free, capable to work at body temperature, and/or wearable either for a day or more.

Many state-of-the-art diagnostic assays for nucleic acid detection rely on measuring the fluorescence intensity of a solution, where the intensity reports on the concentration of fluorescent dyes that emit light upon excitation with a specific wavelength. An advantage of fluorescent read out is high sensitivity, but this sensitivity comes at the expense of requiring instrumentation that is capable of eliciting and detecting the fluorescence signal. The requirement for instrumentation presents an obstacle for widespread point of need applications or home usage of fluorescence-based assays, therefore limiting the scope of fluorescence-based diagnostic (or screening) assays. By contrast, assays that lead to a change of color can in principle be perceived more easily by the naked eye and could thus be better suited for usage at home or in the field. However, as a rule of thumb, absorption-based assays are about 1000-fold less sensitive than fluorescence-based assays. Hence, absorption-based assays are more commonly used for antigen or antibody detection than for nucleic acid based detection, because proteins occur typically at much higher copy numbers than nucleic acids.

A number of fluorescence-based assays for detecting viral nucleic acids use CRISPR technology. At least one case (Fozouni P, Son S, Diaz de Leon Derby M, Knott G J, Gray C N, et al. 2020. Cell) specifically uses the Cas13 protein without a preamplification step. Cas13 is a protein that targets single-stranded (ss) RNA substrates. Cas13 is complexed with a so-called CRISPR RNA (crRNA) containing a programmable spacer sequence to form a nuclease-inactive ribonucleoprotein complex (RNP). When the RNP binds to a complementary target RNA, it activates the HEPN (higher eukaryotes and prokaryotes nucleotide-binding domain) motifs of Cas13 that then indiscriminately cleave any surrounding ssRNAs. Target RNA binding and subsequent Cas13 cleavage activity can be detected with a fluorophore-quencher pair linked by an ssRNA, which fluoresces after cleavage by active Cas13. In the work by Fozouni et al, the reporter ssRNA was 5′-FAM-rUrUrUrUrU-IowaBlack FQ-3′, where FAM is a fluorophore and IowaBlack FQ is a quencher. Other assays use the Cas12 protein, which cleaves single- or double-stranded DNA upon complexation of a target nucleic acid, and which can be employed analogously to the Cas13.

The Cas13 assay is reasonably sensitive, its limit of detection was shown to be about 100 copies of the target RNA per μl. The Cas13 assay thus appears attractive for detecting pathogen nucleic acids in human body fluids, or on surfaces etc. However, the fluorescent readout requires instrumentation for detection.

Designs employing peroxidases to provide readouts signals present their own challenges. First, the function of split G quadruplex peroxidase probes is strongly dependent on the immediate sequence context (see, e.g., Connelly R P, Verduzco C, Farnell S, Yishay T, Gerasimova Y V. 2019. ACS Chem Biol 14:2701-12). Because the G quadruplex strand segments are directly neighboring the detector sequence segments, leaving room for interferences, such systems would benefit from a negative design to exclude potentially inhibitory sequence motifs and to exclude sequence motifs that can generate undesired background activity. Additionally, conventional probes only generate one active peroxidase unit per one target strand. Given limited catalytic activity and background activity, the practical limit of detection is too high (>10 nM) for uses in, e.g., viral nucleic acid detection, consumer genetic testing, etc.

Hence, it is desirable to physically separate the catalytic active sequence motifs from the detector sequence motifs, with inert spacer material in between and to activate multiple peroxidase units per binding event of one target nucleic acid strand.

### BRIEF SUMMARY OF INVENTION

The present system or method disclosed herein may be directed towards a system and methods for detecting the presence of a target analyte such as a specific nucleic acid or a small molecule. The system or method further may include a method of signal amplification and/or a readily understandable readout.

In at least one aspect the invention is a system comprising a translation module, an amplification module, and a detection module. In some embodiments, the translation module comprises a first nucleic acid comprising a first polynucleotide, and a second nucleic acid comprising a second polynucleotide. In some embodiments, the second polynucleotide is configured to reversibly hybridize the first polynucleotide. In some embodiments, the translation module is configured to accept one or more input signals, the one or more input signals comprise one or more target nucleic acids, and the one or more target nucleic acids comprise a target polynucleotide. In some embodiments, the first polynucleotide is configured to hybridize the target polynucleotide, and the second polynucleotide is configured to dissociate from the first polynucleotide to provide a translator output. In some embodiments, the amplification module comprises one or more sensing modalities. In some embodiments, the amplification module is configured to accept the translator output. In some embodiments, the one or more sensing modalities are configured to detect the translator output. In some embodiments, the one or more sensing modalities are configured to provide an amplifier output upon detecting the translator output. In some embodiments, the detection module is configured to accept the amplifier output. In some embodiments, the detection module comprises one or more substrates and one or more activatable nucleic acid peroxidases. In some embodiments, the one or more activatable nucleic acid peroxidases are configured to be converted into one or more active nucleic acid peroxidases in the presence of the amplifier output. In some embodiments, the one or more active nucleic acid peroxidases are configured to convert the one or more substrates into one or more products.

In some embodiments of any of the systems, the one or more sensing modalities comprises one or more of: one or more isothermal chemical ligation-hybridization and chemical cross replication modules configured to replicate the translator output as the amplifier output, one or more hairpin-chain reaction modules configured to replicate the translator output as the amplifier output, one or more duplicator gate cascade modules configured to replicate the translator output as the amplifier output, one or more CRISPR-Cas13 modules configured to provide an active Cas13 complex as the amplifier output, one or more CRISPR-Cas12 modules configured to provide an active a Cas12 complex as the amplifier output, or one or more nucleated polymerization module configured to provide a polymer as the amplifier output.

In some embodiments of any of the systems, the one or more activatable nucleic acid peroxidases are one or more of: one or more caged nucleic acid peroxidases comprising a digestible region, one or more nucleic acid peroxidases in tension, and/or one or more first peroxidase polynucleotides, and one or more second peroxidase polynucleotides. In some embodiments, the amplifier output is configured to digest the digestible region of the one or more caged nucleic acid peroxidases, and the one or more caged nucleic acid peroxidases are configured to be converted to the one or more active nucleic acid peroxidases upon digestion of the digestible region. In some embodiments, the amplifier output is configured to relax the one or more nucleic acid peroxidases in tension, and the one or more nucleic acid peroxidases are configured to be converted to the one or more active nucleic acid peroxidases upon relaxation. In some embodiments, the one or more first peroxidase polynucleotide and the one or more second peroxidase polynucleotide are configured to form the one or more active nucleic acid peroxidases when the one or more first peroxidase polynucleotide are in proximity to the one or more second peroxidase polynucleotide. In some embodiments, the one or more activatable nucleic acid peroxidases are configured to bring the one or more first peroxidase polynucleotides and the one or more second peroxidase nucleotides into proximity when the amplifier output is present.

In some embodiments of any of the systems, the one or more sensing modalities comprises one or more of one or more CRISPR-Cas13 modules configured to provide an active Cas13 complex as the amplifier output or one or more CRISPR-Cas12 modules configured to provide an active Cas12 complex as the amplifier output; the one or more activatable nucleic acid peroxidases are one or more first peroxidase polynucleotides, and one or more second peroxidase polynucleotides. In some embodiments, the one or more first peroxidase polynucleotide and the one or more second peroxidase polynucleotide are configured to form the one or more active nucleic acid peroxidases when the one or more first peroxidase polynucleotide are in proximity to the one or more second peroxidase polynucleotide. In some embodiments, the detection module is configured to bring the one or more first peroxidase polynucleotides and the one or more second peroxidase nucleotides into proximity when the amplifier output is present.

In some embodiments of any of the systems, the system further comprising one or more reporter nucleic acids. In some embodiments, the one or more reporter nucleic acids are configured to hold the one or more first peroxidase polynucleotides separate from the one or more second peroxidase polynucleotides when the one or more reporter nucleic acid is not cleaved. In some embodiments, the one or more activatable nucleic acid peroxidases are configured to bring the one or more first peroxidase polynucleotides into proximity with the one or more second peroxidase polynucleotides when the one or more reporter nucleic acids is cleaved. In some embodiments, the amplifier output is configured to cleave the one or more reporter nucleic acids.

In some embodiments of any of the systems, the system comprises a plurality of activatable nucleic acid peroxidases. In some embodiments, each activatable nucleic acid peroxidase comprises one of the one or more first peroxidase polynucleotide and one of the one or more second peroxidase polynucleotide. In some embodiments, the detection module comprises: a first portion comprising a first surface, the one or more first peroxidase polynucleotides attached to the first surface, a second portion comprising a second surface, the one or more second peroxidase polynucleotides attached to the second surface, and a hinge connecting the first portion and the second portion. In some embodiments, the hinge is configured to hold the detection module in an open form when the one or more reporter nucleic acids is uncleaved or the one or more reporter nucleic acids is single stranded, and the hinge is configured to hold the detection module in a closed form when the one or more reporter nucleic acids is cleaved or the one or more reporter nucleic acids is double stranded. In some embodiments, the one or more first surface and the second surface are configured to separate the one or more first peroxidase polynucleotides and the one or more second peroxidase polynucleotides when the detection module is in the open form, and the first surface and the second surface are configured to bring the one or more first peroxidase polynucleotides into proximity with the one or more second peroxidase polynucleotides when the detection module is in the closed form.

In some embodiments of any of the systems, the one or more sensing modalities comprises one or more of one or more CRISPR-Cas13 modules configured to provide an active Cas13 complex as the amplifier output or one or more CRISPR-Cas12 modules configured to provide an active a Cas12 complex as the amplifier output; and the one or more activatable nucleic acid peroxidases are one or more nucleic acid peroxidases in tension. In some embodiments, the amplifier output is configured to relax the one or more nucleic acid peroxidases in tension, and the one or more nucleic acid peroxidases are configured to be converted to the one or more active nucleic acid peroxidases upon relaxation. In some embodiments, the system further comprises one or more reporter nucleic acids. In some embodiments, the one or more reporter nucleic acids are configured to hold the one or more nucleic acid peroxidases in tension when the one or more reporter nucleic acid is not cleaved, and the amplifier output is configured to cleave the one or more reporter nucleic acids. In some embodiments, the system comprises a plurality of activatable nucleic acid peroxidases, and the detection module comprises a first portion comprising a first surface, the one or more nucleic acid peroxidases are attached to the first surface; a second portion comprising a second surface, the one or more nucleic acid peroxidases are attached to the second surface; and a hinge connecting the first portion and the second portion, the hinge is configured to hold the detection module in an open form when the one or more reporter nucleic acids is uncleaved, and the hinge is configured to hold the detection module in a closed form when the one or more reporter nucleic acids is cleaved. In some embodiments, the one or more first surface and the second surface are configured to hold the one or more nucleic acid peroxidases in tension when the detection module is in the open form, and the first surface and the second surface are configured to hold the one or more nucleic acid peroxidases in tension when the detection module is in the closed form.

In some embodiments of any of the systems, the amplification module further comprises one or more activatable elements. In some embodiments, the amplifier output is configured to activate the one or more activatable elements to provide one or more activated elements. In some embodiments, the one or more activated elements are configured to detect one or more nucleic acids, and the one or more sensing modalities are configured to provide a second amplifier output upon detecting the one or more nucleic acids; and the detection module is further configured to accept the second amplifier output, and the one or more activatable nucleic acid peroxidases are configured to be converted into the one or more active nucleic acid peroxidases in the presence of the second amplifier output. In some embodiments, the one or more activatable elements is a masked nucleic acid, and the one or more activated elements is not masked. In some embodiments, the second amplifier output and the amplifier output are the same. In some embodiments, the one or more activated elements and the translator output are the same.

In some embodiments of any of the systems, the one or more input signals is provided by dissociating the one or more target nucleic acids of the one or more input signals from another polynucleotide.

In at least one aspect the invention is a method for detecting one or more target nucleic acids, the one or more target nucleic acids comprises a target polynucleotide. In some embodiments, method comprises: providing a sample to a translation module, the translation module comprising: a first nucleic acid comprising a first polynucleotide, and a second nucleic acid comprising a second polynucleotide, the second polynucleotide is configured to reversibly hybridize the first polynucleotide; hybridizing the first polynucleotide to the target polynucleotide; dissociating the second polynucleotide from the first polynucleotide to provide a translator output; providing the translator output to an amplification module, the amplification module comprises one or more sensing modalities; detecting the translator output by the one or more sensing modalities; providing an amplifier output from the one or more sensing modalities; providing the amplifier output to a detection module, the detection module comprises one or more substrates and one or more activatable nucleic acid peroxidases; converting the one or more activatable nucleic acid peroxidases into one or more active nucleic acid peroxidases; and converting the one or more substrates into one or more products with the one or more active nucleic acid peroxidases.

In some embodiments of any of the methods, the step of providing the amplifier output from the one or more sensing modalities comprises one or more of: replicating the translator output by isothermal chemical ligation-hybridization and chemical cross replication, replicating the translator output by hairpin-chain reaction, replicating the translator output with duplicator gate cascades, providing an active Cas13 complex, providing an active a Cas12 complex, or providing a polymer by nucleated polymerization.

In some embodiments of any of the methods, the step of converting the one or more activatable nucleic acid peroxidases into one or more active nucleic acid peroxidases comprises one or more of: digesting a digestible region of one or more caged nucleic acid peroxidases, relaxing one or more nucleic acid peroxidases in tension, or bringing one or more first peroxidase polynucleotides and one or more second peroxidase polynucleotides into proximity.

In some embodiments of any of the methods, the step of providing the amplifier output from the one or more sensing modalities comprises providing one or more of an active Cas13 complex and an active a Cas12 complex; and the step of converting the one or more activatable nucleic acid peroxidases into one or more active nucleic acid peroxidases comprises one or more of one or more first peroxidase polynucleotides and one or more second peroxidase polynucleotides into proximity. In some embodiments, the step of converting the one or more activatable nucleic acid peroxidases into one or more active nucleic acid peroxidases comprises cleaving one or more reporter nucleic acids with the active Cas13 complex or active Cas12 complex, and the one or more reporter nucleic acids are configured to hold the one or more first peroxidase polynucleotides separate from the one or more second peroxidase polynucleotides when the one or more reporter nucleic acid is not cleaved.

In some embodiments of any of the methods, the step of providing the amplifier output from the one or more sensing modalities comprises providing one or more of an active Cas13 complex and an active a Cas12 complex; and the step of converting the one or more activatable nucleic acid peroxidases into one or more active nucleic acid peroxidases comprises relaxing one or more nucleic acid peroxidases in tension. In some embodiments, the step of converting the one or more activatable nucleic acid peroxidases into one or more active nucleic acid peroxidases comprises cleaving one or more reporter nucleic acids with the active Cas13 complex or active Cas12 complex, and the one or more reporter nucleic acids are configured to hold the one or more activatable nucleic acid peroxidases in tension when the one or more reporter nucleic acid is not cleaved.

In some embodiments of any of the methods, the amplification module further comprises one or more activatable elements, and the method further comprises: activating the one or more activatable elements with the amplifier output, providing one or more activated elements, detecting one or more nucleic acids with the one or more activated elements, providing a second amplifier output, and providing the second amplifier output to the detection module. In some embodiments, the one or more activatable elements is a masked nucleic acid, and the one or more activated elements is not masked. In some embodiments, the second amplifier output and the amplifier output are the same. In some embodiments, the one or more activated elements and the translator output are the same.

In some embodiments of any of the methods, the method further comprises dissociating the one or more target nucleic acids of the one or more input signals from another polynucleotide.

### BRIEF DESCRIPTION OF THE DRAWINGS

FIG. 1 depicts an exemplary embodiment of a hinged system sensitive to nucleic acid strandedness.

FIG. 2 depicts an exemplary embodiment of a hinged system sensitive to nucleic acid strandedness.

FIG. 3 depicts an exemplary embodiment of a switchable hinged-beam nucleic acid nanodevice with a stretched (left) and relaxed (right) peroxidase.

FIG. 4 depicts an exemplary embodiment of a switchable hinged-beam nucleic acid nanodevice with a split peroxidase separated (left) and in proximity to one another (right).

FIG. 5 depicts an exemplary embodiment of a switchable hinged-beam nanodevice where the inactive state (left) possesses an uncleaved double-stranded nucleic acid and the active state (right) possesses a cleaved double-stranded nucleic acid.

FIG. 6 depicts a molecular rendering of a DNA holiday junction.

FIGS. 7A-7B depict exemplary embodiments of a switchable hinged system possessing multiple peroxidases. FIG. 7A depicts the exemplary embodiment with an uncleaved reporter nucleic acid and stretched peroxidases. FIG. 7B depicts the exemplary embodiment a cleaved reporter nucleic acid and relaxed peroxidases.

FIGS. 8A-8E depict an exemplary embodiment comprising a single nucleic acid strand including a peroxidase sequence. The peroxidase may be inhibited by complementary regions and/or the formation (FIG. 8A). A hairpin loop region may be cleaved by a nuclease (FIG. 8B) to produce an active peroxidase unit (FIG. 8C). FIG. 8D depicts stability of an exemplary hairpin sequence. FIG. 8E depicts an exemplary embodiment activated by the introduction of an invader strand. FIG. 8A discloses SEQ ID NOS 6, and 1, FIG. 8B discloses SEQ ID NOS 6 and 1, FIG. 8C discloses SEQ ID NOS 6, and 1, FIG. 8D discloses SEQ ID NOS 7, 6, and 8, and FIG. 8E discloses SEQ ID NOS 9, and 1, all respectively, in order of appearance.

FIG. 9 depicts an exemplary embodiment of a G-plex (peroxidase) stretcher. FIG. 9 discloses SEQ ID NOS 11-13, and 10, respectively, in order of appearance.

FIGS. 10A-10B depict exemplary embodiments of a circularized G-plex (peroxidase) stretcher. FIG. 10A discloses SEQ ID NOS 14-17, and FIG. 10B discloses SEQ ID NOS 15, 14, 17, 16, and 61, all respectively, in order of appearance.

FIGS. 11A-11E depict an exemplary embodiment of constrained G-plex (peroxidase) variants. FIG. 11A discloses SEQ ID NOS 18, 20, and 19, FIG. 11B discloses SEQ ID NOS 21, 23, and 22, FIG. 11C discloses SEQ ID NOS 18, 20, and 24, FIG. 11D discloses SEQ ID NOS 21, 23, and 25, FIG. 11E discloses SEQ ID NOS 61, 1, 26, 62, 18, 27, 63, 21, 26, 64, 18, 27, 41, 21, 26, 28, 18, 27, 29, 21, 26, 30, 18, 27, 31, and 21, all respectively, in order of appearance.

FIGS. 12A-12B depicts an exemplary embodiment of a G-plex (peroxidase) inhibited by stretching. FIG. 12A discloses SEQ ID NOS 33-35 and 32, and FIG. 12B discloses SEQ ID NOS 61, 1, 34-35 and 32-33, all respectively, in order of appearance.

FIG. 13 depicts an exemplary embodiment of a G-plex (peroxidase) inhibited by stretching. FIG. 13 discloses SEQ ID NOS 36-37, respectively, in order of appearance.

FIGS. 14A-14C depict exemplary embodiments of G-plexes (peroxidases) inhibited by stretching. FIG. 14A discloses SEQ ID NOS 38, 40, and 39, FIG. 14B discloses SEQ ID NOS 38, 40 and 39, and FIG. 14C discloses SEQ ID NOS 61, 1, 37, 36, 38, 40, and 42, all respectively, in order of appearance.

FIG. 15 depicts an exemplary embodiment of a card system for detecting an analyte.

FIG. 16 depicts an exemplary embodiment of a card system with a QR code for detecting an analyte.

FIG. 17 depicts an exemplary embodiment of a system for detecting an analyte present on one or more people in a public area.

FIG. 18 depicts an exemplary architecture overview of an exemplary embodiment of a card system for detecting an analyte.

FIG. 19A-19L depict performance modeling of an exemplary double-catalytic system comprising activated Cas13. Solid lines denote a positive output, and dashed lines denote a negative control reaction run in parallel. Stars indicate a reasonable expectation of the limit of detection. The figures assume different peroxidase turnover rates (ku4), Cas13 turnover rates (k_{cas}), background active peroxidase, and analyte concentrations (trigger).

FIG. 20 depicts exemplary data obtained with closed hairpin G4 quadruplex variants (dashed lines) and fully active variants (solid lines).

FIG. 21A-21B depict an exemplary architecture overview of an exemplary embodiment of a card system for detecting an analyte.

FIG. 22 depicts exemplary data obtained with closed hairpin G4 quadruplex variants (solid lines) and fully activated variants (dashed lines).

FIG. 23 depicts exemplary data obtained with caged G4 quadruplex variants (solid lines) and fully active variants (dashed lines).

FIG. 24 depicts exemplary data obtained with stretched G4 quadruplex variants (solid lines) and fully active variants (dashed lines).

FIGS. 25A-25H depict G plex peroxidase duplexes. FIG. 25A discloses SEQ ID NOS 43-44, FIG. 25B discloses SEQ ID NOS 46, 45, and 44, FIG. 25C discloses SEQ ID NOS 48, 47, and 44, FIG. 25D discloses SEQ ID NOS 50, 49, and 44, FIG. 25E discloses SEQ ID NOS 51-52, FIG. 25F discloses SEQ ID NOS 54, 53, and 52, FIG. 25G discloses SEQ ID NOS 56, 55, and 52, and FIG. 25H discloses SEQ ID NOS 58, 57, and 62, all respectively, in order of appearance.

FIG. 26 depicts an exemplary embodiment of an in-solution system.

FIG. 27 depicts an exemplary embodiment including a feedback loop using an auto-catalytic cascade. FIG. 27 discloses SEQ ID NOS 59-60, 1, 60, 1, 60, 1, 59-60, respectively, in order of appearance.

### DETAILED DESCRIPTION

In one aspect, the present system or method is directed towards systems and/or methods for detecting one or more analytes using molecular beacons to provide one or more detectable signals. In some embodiments the detectable signal is provided by use of a peroxidase that is configured to switch from an inactive state to an active state. In some embodiments there is a many-to-one correspondence between each molecule contributing to the detectable signal and each molecule of the analyte. In some embodiments, each molecule of the analyte corresponds to multiple molecules contributing to the detectable signal, e.g., each molecule of the analyte may correspond to two, tens, hundreds, thousands, or tens of thousands of molecules contributing to the detectable signal.

An analyte can be any detectable molecule of interest including but not limited to a nucleic acid and a small molecule. In some embodiments, the analyte is a nucleic acid, for example a DNA or an RNA. An analyte may be obtained from any appropriate source including, but not limited to, saliva, exhalation, sweat, the skin microbiome, or an object's surface. An analyte may be extracted by any suitable method known in the art. For example, extraction of a nucleic acid may be achieved by including a lysis buffer such as 10% protease K, 0.7 M NaCl, 0.1% Hexadecyl trimethyl ammonium Bromide (CTAB) and MES at pH 5.0. Other methods known in the art suitable for nucleic acid extraction are contemplated.

The sample may be prepared in any suitable way. In some embodiments, the methods or systems directly accept analytes (e.g., nucleic acids) from bodily fluids such as saliva. In some embodiments, DNA and RNA nucleases present in a raw sample or other external sources may be deactivated. In some embodiments, the sensor spot contains a sufficiently high concentration of broad-band nuclease inhibitors to efficiently deactivate nucleases. In some embodiments, the sensor contains two initially physically separate sensor areas. The first area comprises a hydrogel matrix containing sufficiently high concentrations of proteinase K to deactivate nucleases. In some embodiments, a sample (e.g., saliva) is applied directly to the hydrogel matrix of the first area.

In some embodiments, a sample (e.g., saliva) is applied by first unpeeling a cover which will expose the application spot to air. Oxygen in the air will activate an exothermic chemical reaction in a heat pad layer positioned underneath the sample application spot. In some embodiments, the heat pad is configured to locally heat the area underneath the sample application spot to an appropriate temperature. In some embodiments, the temperature is approximately 90° C. In some embodiments, proteins present in a sample (e.g., saliva) applied to the application spot will be deactivated by proteinase K provided by the form. In some embodiments, the proteinase K will be denatured by the heating reaction. In some embodiments, after a suitable period of time, the processed sample present at the first area is provided to the second area. In some embodiments, a suitable period of time is approximately 10 minutes. In some embodiments, the second area comprises sensor components (e.g., modules or layers).

In some embodiments, the analyte may undergo a pre-detection amplification step such as whole genome amplification. This amplification increases the amount of a genome (e.g., a viral genome) available that may possess the analyte of interest. Such whole genome amplification may increase the likelihood that there is sufficient analyte available to generate detectable signal without the use of an additional instrument (e.g., by the naked eye). Exemplary whole genome amplification systems may be based on Phi29 or any known polymerase. In some embodiments, the polymerase is isothermal and is enzymatically active at skin temperature (e.g., Phi29).

In another aspect, the generated signal undergoes an exponential amplification for subsequent detection. In some embodiments, no analyte amplification step (e.g., whole genome amplification) may be needed. In some embodiments, an analyte amplification step may precede signal amplification.

In at least one aspect the present system or method comprises a translation module, an amplification module comprising one or more sensing modalities, and a detection module configured to accept the amplifier output.

In some embodiments, the system or method is capable of detecting trace amounts of nucleic acid molecules with high sensitivity and specificity in a device-free system with naked-eye readable output. In some embodiments, they system or method uses a nuclease that can be programmatically activated to produce indiscriminate nuclease activity upon the initial detection of a specific nucleic acid sequence. In some embodiments the system or method is auto-catalytic.

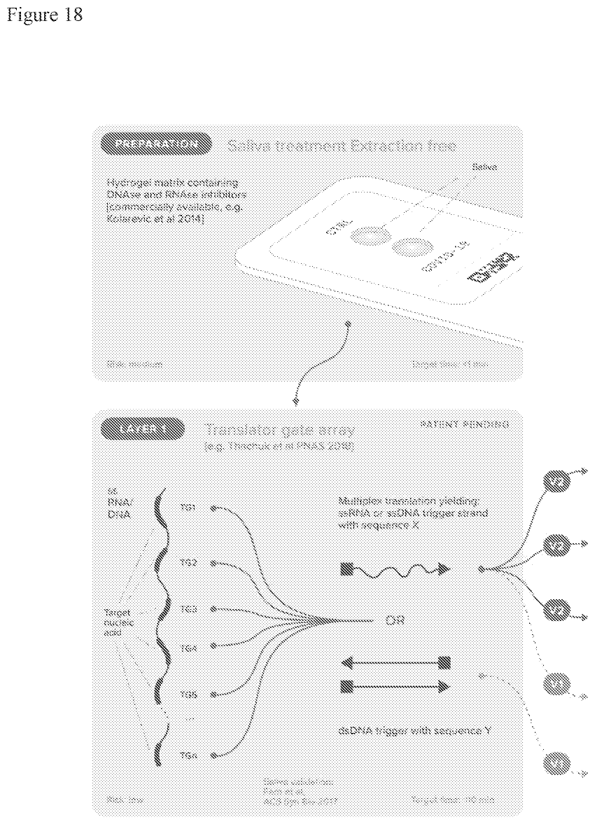

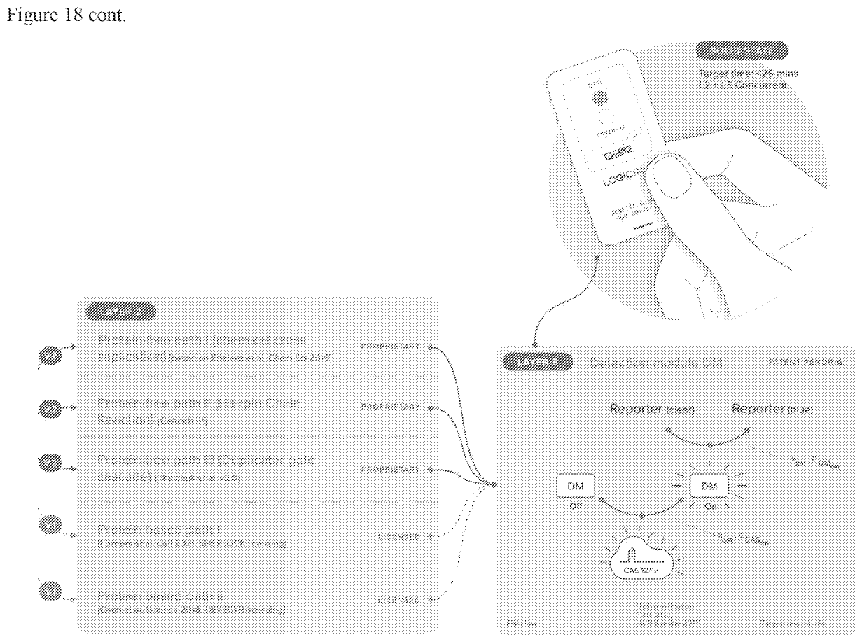

In preferred embodiments, the form factor is a modular system for naked-eye readable nucleic acid detection. In some embodiments, the system comprises three signal amplification layers. For example, FIGS. 18 and 21A-21B depict an architecture overview of an exemplary sensor as exemplified in a card-like form factor.

### Translation Module

In some embodiments, the translation module comprises a first nucleic acid comprising a first polynucleotide, and a second nucleic acid comprising a second polynucleotide, wherein the second polynucleotide is configured to reversibly hybridize the first polynucleotide. In some embodiments, the translation module is configured to accept one or more input signals. In some embodiments, the one or more input signals comprise one or more target nucleic acids, wherein the one or more target nucleic acids comprise a target polynucleotide. In some embodiments, the first polynucleotide is configured to hybridize the target polynucleotide, and the second polynucleotide is configured to dissociate from the first polynucleotide to provide a translator output (e.g., an activating or first activating sequence).

In some embodiments, the plurality of nucleic acid input signals includes at least 1 target, at least 2 targets, at least 5 targets, at least 10 targets, at least 25 targets, at least 50 targets, at least 75 targets, at least 100 targets, at least 250 targets, at least 500 targets, at least 750 targets, or at least 1,000 targets. In some embodiments, the plurality of nucleic acid input signals includes one or more targets of a viral or bacterial nucleic acid. In some embodiments, the plurality of nucleic acids of the system are derived from the genome of SARS-CoV-2.

In some embodiments, the translator module employs DNA strand displacement techniques. In some embodiments, a translator gate binds to a unique genetic sequence (ssDNA or ssRNA). In some embodiments, the translator gate is configured to output a ssRNA or ssDNA trigger strand with sequence X. in some embodiments, the translator gate is configured to output a double-stranded (ds) DNA trigger or dsRNA trigger with sequence Y. In some embodiments, the translator gate is a nucleic acid (e.g., a first nucleic acid) and the output trigger is a second nucleic acid.

An array of unique translator gates can be formed, each targeting different regions along a genome of interest. Arrays may be configured to increase the production of trigger strands X or Y. Wang B, Thachuk C, Ellington A D, Winfree E, Soloveichik D. 2018. Proc Natl Acad Sci USA 115:E12182-E91.

In some embodiments, the system of method may be altered to detect different or new analytes only by altering the translation module

In some embodiments, the translation module is the first layer of a multilayer architecture.

In some embodiments, translator output is an RNAse Cas13 configured to cleave an RNA component of the activatable peroxidase thereby activating (e.g., ungating) the peroxidase in the detector layer. In some embodiments, translator output is an DNAse Cas12 configured to cleave an DNA component of the activatable peroxidase thereby activating (e.g., ungating) the peroxidase in the detector layer.

### Amplification Module

In some embodiments, the amplification module is configured to accept the translator output (e.g., the activating or first activating sequence). In some embodiments, the one or more sensing modalities are configured to detect the translator output, and the one or more sensing modalities are configured to provide an amplifier output (e.g., an active or first active nuclease) upon detecting the translator output. In some embodiments, the amplifier output (e.g., an active or first active nuclease) is an active Cas protein (e.g., an active Cas13 protein). In some embodiments, an inhibited nuclease or first inhibited nuclease is activated to provide the amplifier output (e.g., an active or first active nuclease). In some embodiments, the inhibited nuclease or first inhibited nuclease is an inactive Cas protein, inactive Cas13 protein, or inactive CRISPR-Cas13 complex and the amplifier output (e.g., an active or first active nuclease) is an active Cas protein, active Cas13 protein, or active CRISPR-Cas13 complex.

In some embodiments, the amplification module further comprises one or more activatable elements. In some embodiments the one or more activatable elements are one or more masked activatable nucleic acid sequences. In some embodiments, the one or more masked activatable nucleic acid sequences may possess one or more cryptic or hidden recognition sequences or strands. For example, the one or more cryptic or hidden recognition (i.e., masked) strands or sequences may be masked such that a Cas protein (e.g., Cas13) is unable to recognize the strand or sequence, bind to the strand or sequence, and become active. If the sequence or strand is unmasked or released it is no longer cryptic or hidden. Such unmasked strands or sequences may then be recognized by a Cas protein (e.g., Cas13), bind to the protein, and the protein is able to become active. In some embodiments, the amplifier output (e.g., an active or first active nuclease) is configured to activate the one or more activatable elements to provide one or more activated elements (e.g., an activating or second activating sequence). In some embodiments, the one or more activated elements (e.g., an activating or second activating sequence) are configured to detect one or more nucleic acids. In some embodiments, the one or more sensing modalities are configured to provide a second amplifier output (e.g., an active or second active nuclease) upon detecting the one or more nucleic acids. In some embodiments the second amplifier output (e.g., an active or second active nuclease) and the amplifier output (e.g., an active or first active nuclease) are the same. In some embodiments, the one or more activated elements (e.g., an activating or second activating sequence) and the one or more input signals are the same. In some embodiments, the one or more activated elements (e.g., an activating or second activating sequence) and the translator output (e.g., an activating or first activating sequence) are the same.

In some embodiments, the amplifier output comprises a nuclease that is activated upon binding an ssDNA, an ssRNA, a dsDNA or a dsRNA translator output (e.g., from the translator layer). In some embodiments, the amplifier output comprises one or more of; one or more isothermal chemical ligation-hybridization and chemical cross replication modules configured to replicate the translator output (see, e.g., Edeleva et al, Chem. Sci., 2019, 10, 5807-5814), one or more hairpin-chain reaction modules configured to replicate the translator output (see, e.g., Harry et al., ACS Nano 2014, 8, 5, 4284-4294), one or more duplicator gate cascade modules configured to replicate the translator output (see, e.g., Thatchuk et al, v2.0), one or more CRISPR-Cas13 modules configured to provide an active Cas13 complex (see, e.g, Fozouni et al., 2021, Cell 184, 323-333), one or more CRISPR-Cas12 modules configured to provide an active a Cas12 complex (see, e.g., Chen, Janice S., et al. “CRISPR-Cas12a target binding unleashes indiscriminate single-stranded DNase activity.” Science 360.6387 (2018): 436-439), and/or one or more nucleated polymerization module configured to provide a polymer (see, e.g., Minev, Dionis, et al. “Robust nucleation control via crisscross polymerization of DNA slats.” bioRxiv (2019)).

In some embodiments, the amplifier output comprises one or more duplicator gate cascade modules, wherein a set of DNA strands is configured such that a single signal (e.g., an input signal nucleic acid) can hybridized the duplicator gate. The hybridization of the signal to the duplicator gate promotes dissociation of two or more of the strands of the duplicator gate resulting in an amplified output signal. In some embodiments, the output signal comprises different sequences. In some embodiments, the output signal comprises the same sequence.

In some embodiments, the amplification module is the second layer of a multilayer architecture.

In some embodiments, the amplifier layer comprises a nuclease. In some embodiments the nuclease of the amplifier layer is and RNAse. In some embodiments, the RNAse is RNAse A. In some embodiments, the RNAse A is configured to be activated by the translator output. In some embodiments, the RNAse A is coupled to a nucleic acid configure to allow its activation by the translator output. In some embodiments, the RNAse of the amplifier layer is coupled to a surface and it is configured to be activated when released from the surface by nucleic acid strand displacement.

In some embodiments, the amplification layer of the system comprises a nucleic acid circuit. In some embodiments, the nucleic acid circuit is configured to create an amplifier output upon addition of a translator output. In some embodiments, the amplifier output is one or more ssRNA, ssDNA, or partially duplex species. Partially duplex species include dsDNA, dsRNA, or dsDNA/RNA hybrids. In some embodiments, the nucleic acid circuit of the system creates an amplifier output that is at least 10 times the concentration of the translator output. In some embodiments, the nucleic acid circuit of the system creates an amplifier output that is at least 100 times the concentration of the translator output. In some embodiments, the nucleic acid circuit of the system creates an amplifier output that is at least 1000 times the concentration of the translator output.

In some embodiments, the amplification module as the second layer responds to the output from the translator module as the first layer. In some embodiments, the second layer is responsive to one or more of a ssDNA, a ssRNA, a dsDNA, a dsRNA, or a hybrid dsDNA/RNA molecule with specified sequence from the first layer (e.g., the translator output or trigger).

In some embodiment, the second layer comprises a plurality of sensing modalities. The sensing modalities may be distinct. At least one or more of the sensing modalities is responsive to the trigger. These modalities can be used individually, in parallel, or in sequence.

In some embodiments, the second layer uses a protein free nucleic-acid strand amplification cascade responsive to the trigger as a sensing modality. In some embodiments, the amplification cascade is configured to output an amplified signal (amplified output). In some embodiments, the amplified output is one or more of a ssDNA, a ssRNA, a dsDNA, a dsRNA or a hybrid dsDNA/RNA molecule or motifs. The amplified output may have the same sequence as a trigger or may have a sequence distinct from trigger. In some embodiments, the amplification cascade yields N copies of output molecules or motifs per 1 trigger (input) signal provided to the amplifier module or layer. N may be at least 1, at least 10, at least 100, or at least 1000. The expected N depends on the details of the cascade and the time allotted for running the cascade.

Examples of nucleic-acid strand amplification cascades include so-called hairpin-chain reaction cascades (Choi H M, Beck V A, Pierce N A. 2014. ACS Nano 8:4284-94) and isothermal chemical ligation-hybridization cycle (Edeleva E, Salditt A, Stamp J, Schwintek P, Boekhoven J, Braun D. 2019. Chem Sci 10:5807-14). These approaches can be adapted to amplify any output strand species in response to a trigger strand motif.

In some embodiments, the sensing modality utilizes a CRISPR-Cas based nucleic acid detection. The CRISPR-Cas complex is configured to detect a target nucleic acid molecule using a specifically designed crRNA. In some embodiments, the Cas protein is a Cas13 protein. Upon complexing with the specifically designed crRNA, the Cas13 protein becomes activated and acts as a nuclease that indiscriminately digests single-stranded RNA. Fozouni P, Son S, Diaz de Leon Derby M, Knott G J, Gray C N, et al. 2020. Cell. In some embodiments, the Cas13/crRNA ribonucleoprotein (RNP) complex is designed to respond to trigger which is output from the first layer (e.g., the trigger or translator signal). In some embodiments, each trigger molecule yields an activated Cas13 protein capable of degrading ssRNA molecules with the rate kca. In some embodiments, the Cas13 RNP is designed to respond to an amplified output produced by a nucleic-acid strand amplification cascade provided by the amplification module (e.g., the second layer).

In some embodiments, a Cas12a protein can be utilized. Cas12a indiscriminately digests single-stranded DNA molecules once activated.

In some embodiments, the output of a second layer may be one or more of:
M activated Cas13 or Cas12 molecules per trigger strand produced from the first layer when only using Cas13 or Cas12;
N amplified output molecules or motifs per trigger strand from the first layer when only using a nucleic acid amplification cascade; or
N*M activated Cas13 or Cas12 when the Cas13 or Cas12 RNP are designed to respond to amplified output produced by a nucleic acid amplification cascade.

### Detection Module

In some embodiments the detection module comprises one or more substrates and one or more activatable nucleic acid peroxidases. The one or more substrates and one or more activatable nucleic acid peroxidases may be an activatable colorimetric actuator. In some embodiments, the one or more activatable nucleic acid peroxidases are configured to be converted into one or more active nucleic acid peroxidases in the presence of the amplifier output (e.g., an active or first active nuclease). The one or more substrates and one or more activated nucleic acid peroxidases may be an activated colorimetric actuator. In some embodiments, the one or more active nucleic acid peroxidases are configured to convert the one or more substrates into one or more products.

In some embodiments, the detection module is further configured to accept the second amplifier output (e.g., an active or second active nuclease). In some embodiments, the one or more activatable nucleic acid peroxidases are configured to be converted into the one or more active nucleic acid peroxidases in the presence of the second amplifier output (e.g., an active or second active nuclease).

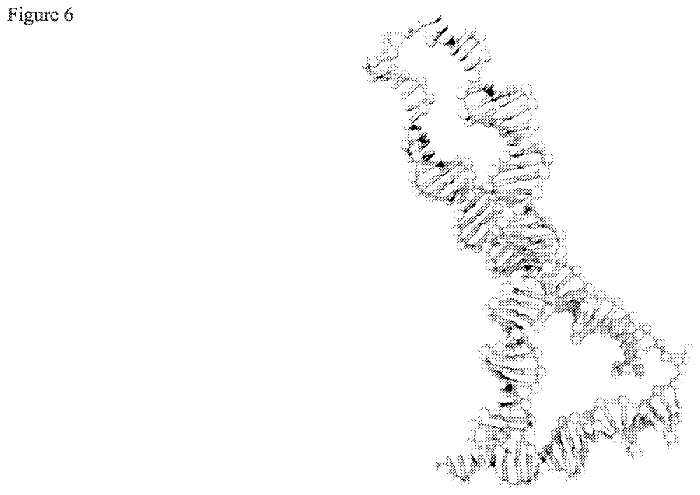

In one aspect, the systems or methods employ one or more switchable nanostructures. In some embodiments, the switchable nanostructure can be constructed from nucleic acids (e.g., DNA and/or RNA). For example, a DNA holiday junction (FIG. 6) is a simple hinged-beam nanodevice. The holiday junction comprises two near vertical DNA helices that are constrained by a separator helix at the helices' lower portions. The constraint transduces strain on a single-stranded DNA motif at the top of the holiday junction.

### Split Probes

In some embodiments, the systems or methods employ enzymatic units split into two inactive subunits that are configured to become active when the two subunits come into proximity with one another. In some embodiments, the split enzymatic units are nucleic acids. In some embodiments, split inactive subunit pair to form a Deoxyribozyme (DNAzyme) or Ribozyme (RNAzyme). In some embodiments, the Deoxyribozyme (DNAzyme) is a peroxidase-mimicking G-quadruplex Deoxyribozyme (DNAzyme) (PDz) that catalyzes generation of a colorimetric signal. Any suitable Deoxyribozyme (DNAzyme) or Ribozyme (RNAzyme) that is capable of producing a detectable signal may be used. The system or method may include any cofactors (e.g., hemin). In some embodiments, the two inactive subunits are portions of a G quadruplex probes that become an active G quadruplex when the subunits come into proximity with one another (e.g., the probes are in a closed state). In some embodiments, the system may contain an integer multiple of a subunit (n) and an integer multiple of a second subunit (m). In such embodiments, the first subunits and the second subunits may be brought into proximity with one another such that an integer multiple of active enzymatic units are formed (x). In some embodiments, x equals n. In some embodiments, x equals m. In some embodiments, n, m, and x are all equal.

In some embodiments, the probes are active when a target nucleic acid stabilizes a closed state of the nanostructures. In a closed state, a pair of subunits will be in proximity to one another. Such a pair may recombine, thereby producing an active enzymatic unit. The pair may be in proximity to one another at any suitable distance that permits recombining of the two paired subunits. In some embodiments the two subunits are in proximity when they are about 0.1 nm to about 20 nm, about 0.5 nm to about 15 nm, about 1 nm to about 10 nm, about 2 nm to about 10 nm, about 1 nm, about 2 nm, about 3 nm, about 4 nm, about 5 nm, about 6 nm, about 7 nm, about 8 nm, about 9 nm, about 10 nm, about 11 nm, about 12 nm about 13 nm about 14 nm, or about 15 nm from one another.

In some embodiments, the immediate neighborhood of an active enzymatic unit (e.g., G-quadruplex) when the nanostructure is in a closed state will be duplex DNA. The neighboring duplex DNA will assist in reducing interference with single-stranded elements.

In some embodiments, one or more subunit of a subunit pair may be a blunt-end subunit. Without being bound by theory, it is believed that switchable or dimerizable DNA nanostructures in which blunt-end DNA interfaces are being brought into proximity with one another assists in increasing positioning accuracy of the subunits. See Gerling T, Wagenbauer K F, Neuner A M, Dietz H. 2015. Science 347:1446-52.

### Signal Amplification

In some embodiments, the systems or methods use signal amplification. In some embodiments, the signal amplification is implemented by nucleated polymerization of nanostructures. In such embodiments, a nucleus form upon addition of a target strand, templating the growth of a filamentous or dendritic structure where upon monomers may be integrated. Each monomer integrated into the filament is configured to generate one or more new active enzymatic unit (e.g., G-quadruplex peroxidase). For example, Minev D, Wintersinger, C. M., Ershova, A., Shih, W. M. 2019. bioRxiv demonstrate nucleated growth of ribbons including triggering the growth of ribbons upon detection of a 192 nt long target nucleic acid motif. In some embodiments, a pair of G-quadruplex subunits are configured as binding domains between the crisscross slat units of the ribbon. Thus, as the ribbon grows, active G-quadruplex units are generated.

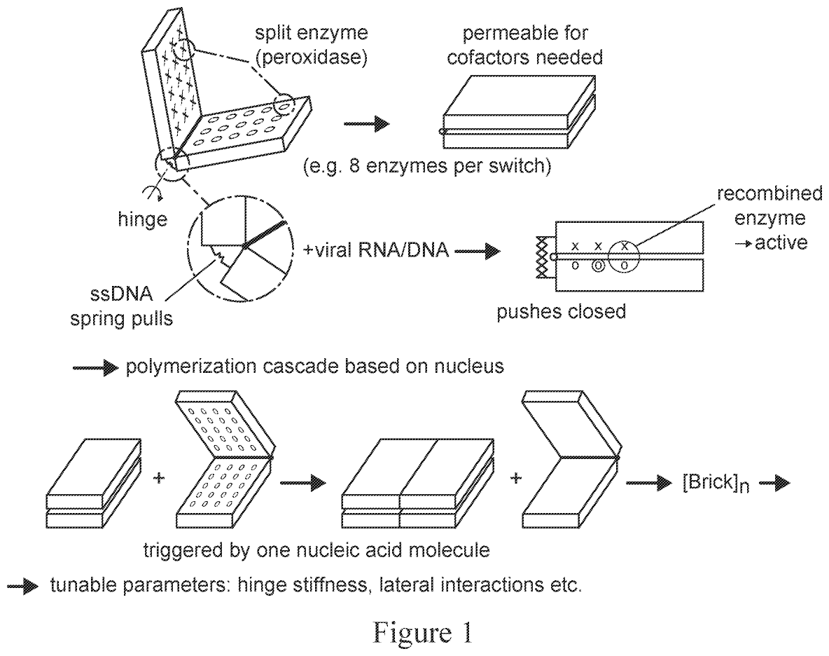

In some embodiments, the system or method comprises two nucleic acid (e.g., DNA) based bricks attached to each other by their edges to form a clamshell structure (e.g., FIGS. 1, 2, and 7A-7B). In some embodiments, the clamshell structure comprises a first portion comprising a first surface with one or more first peroxidase polynucleotides attached, a second portion comprising a second surface with one or more second peroxidase polynucleotides attached, and a hinge connecting the first portion and the second portion. In some embodiments, the hinge is configured to hold the clamshell (i.e., the detection module) in an open (i.e., inactive) form when one or more reporter nucleic acids is uncleaved, and the hinge is configured to hold the clamshell (i.e., the detection module) in a closed (i.e., active) form when the one or more reporter nucleic acids is cleaved. When the clamshell is in an open or inactive form, the one or more first surface and the second surface are configured to separate the one or more first peroxidase polynucleotides and the one or more second peroxidase polynucleotides. When the clamshell is in a closed or active form, the first surface and the second surface are configured to bring the one or more first peroxidase polynucleotides into proximity.

The two bricks may be part of a single DNA structure, or covalently attached through the hinge, or fused in any other ways. In some embodiments, the bricks are constructed using the methods of DNA origami, by other DNA nanotech approaches (e.g., tile assembly), or any other appropriate manner. In some embodiments, each brick is decorated on its interior face (e.g., surface) by multiple segments of a split enzymatic unit (e.g., a G-quadruplex peroxidase).

For example, if A denotes a 3′ segment of a split G-quadruplex peroxidase, and B denotes the 5′ segment of the same split G-quadruplex peroxidase, the first and second surfaces (i.e., the two opposing brick surfaces) may be separately decorated with N copies of A and N copies of B, respectively. In this way, if the clamshell is in a closed state, the A and B subunits are brought into proximity with one another permitting the formation of N active G-quadruplex peroxidases controlled by a single switching event.

Without being bound by theory, the recombination of the G-quadruplexes peroxidases liberates the free energy associated with split G-quadruplexes peroxidases. Hence, the closed state of the brick switch (e.g., the clamshell) may constitute a global free energy minimum. In some embodiments, the clamshell possesses a high energy barrier in its open, inactive state to limit conversion into a closed, active state in the absence of a trigger, kinetically trapping the brick in an open conformation. In some embodiments, the open conformation of the clamshell is limited from converting into a closed, active state in the absence of a trigger using steric occlusion.

In some embodiments, electrostatic repulsion between the like-charged polyanionic surfaces of the brick switch may constitute a force that counteracts closure in the absence of a trigger. In some embodiments, a molecular, spring-loaded hinge mechanism may counteract closure in the absence of a trigger. Funke J J, Ketterer P, Lieleg C, Schunter S, Korber P, Dietz H. 2016. Sci Adv 2:e160097. In some embodiments, single or multiple single-stranded DNA springs may connect the bricks exerting similar hinge edges, thereby maintaining the clamshell in the open form in the absence of a trigger. In some embodiments, the spring single-stranded DNA may further comprise secondary structure elements such as a hairpin motif In some embodiments, the clamshell's one or more springs provides a force to pulling each brick surface away from each other in its inactive state. Accordingly, the clamshell is prevented from closing and activating the split enzymatic units decorating the interior surfaces of the bricks. Without being bound to theory, the free energy stored in a hairpin motif present in a single-stranded DNA spring would add to the kinetic barrier for closing an inactive clamshell because the hairpin would need to be opened (i.e., the complementary base pairs of the hairpin would need to be melted or dissociated from one another) in order to allow for closing the device.

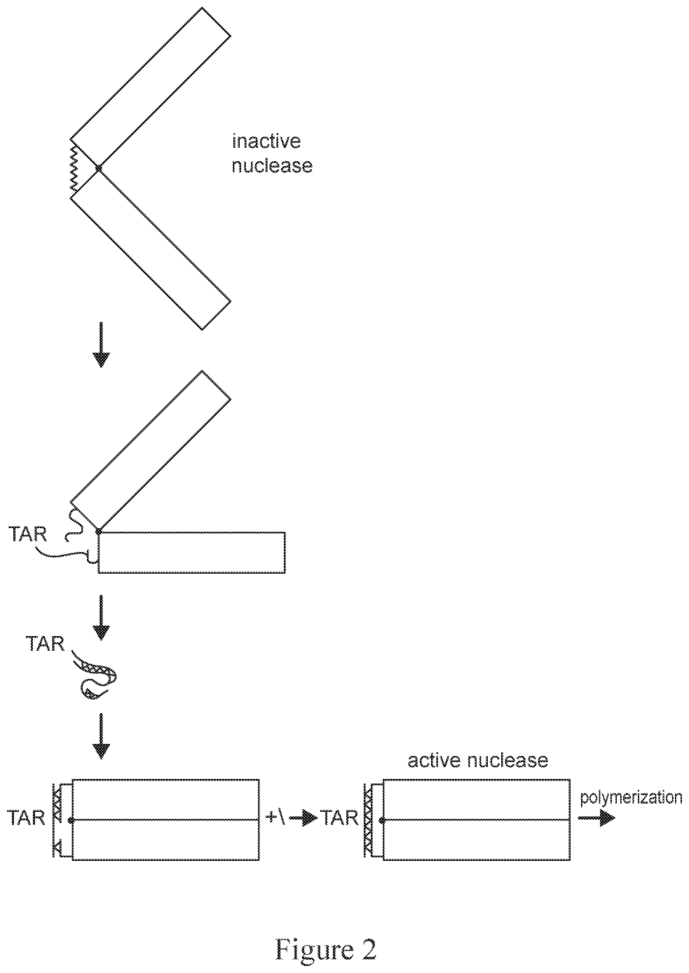

In some embodiments, a target nucleic acid interacts directly with the spring single-stranded DNA strands by, for example, forming a straight duplex DNA (FIGS. 1 and 2). In some embodiments, the formation of a duplex DNA resolves a hairpin motif of the single-stranded spring DNA. In some embodiments, the duplex DNA allows for or promotes the bricks of the clamshell to form a closed clamshell configuration. In the closed configuration, the paired enzymatic subunits (e.g., G-quadruplex peroxidase segments) separated on the two interior faces of the clamshell are brought into proximity to one another.

Further, in some embodiments, the bricks are designed to be permeable to cofactors, substrates, and/or other molecules. Such cofactors, substrates, and/or other molecules may promote or be necessary for the active enzymatic unit (e.g., G-quadruplex peroxidase) to perform its enzymatic function (e.g., its peroxidase function). In some embodiments, a closed clamshell with active enzymatic units (e.g., G-quadruplex peroxidases) is configured as a seed nucleus. In some embodiments, the active form of the clamshell templates the formation of filaments configured to recombine integer multiples of enzymatic units (e.g., G-quadruplex peroxidases) to form per monomer integration into the filament.

In some embodiments, multiple clamshells may be configured to cause amplification of the signal from a trigger strand through a cascade. For example, in some embodiments, closure of a clamshell structure leads to the display of one or more DNA motifs. In some embodiments, the deactivating DNA motif is formed upon clamshell closure through recombination of fully complementary DNA duplex, thereby releasing one or more DNA motifs holding the clamshell opened. These DNA motifs are configured to deactivate the spring system on a second clamshell brick in the open and inactive form. This deactivation removes of the kinetic barrier preventing closure of the second clamshell and permits it to convert into a closed and active form. The closed form of the second clamshell may further display one or more DNA motifs configured to deactivate the spring system of additional open and inactive clamshell. Thus, the systems and methods may use any number of clamshells in a signaling cascade. In embodiments employing such systems, the number of active enzymatic units (e.g., G-quadruplex peroxidases) may be increased by many multiples in the presence of a single copy of the target strand.

The spring mechanism of any of the clamshell configurations disclosed herein, may be provided in a number of ways. Methods in addition to those mentioned above, may include using DNA toehold and strand displacement techniques. In such configurations, the initial inactive spring (S) is displaced on one of its ends by a target strand. The target sequence hybridizes with S to form a longer spring (S′) that is partially duplex. The single stranded gap is hybridized by a smaller complementary strand that is present in the system, forming a more stable and stronger duplex spring (S″) and adding further stability to the closed clamshell state. In some embodiments, the use of DNA toehold and strand displacement is combined with nucleus polymerization as described above.

In some embodiments, the nucleic acid peroxidases of the systems or methods is a DNAzyme called peroxidase-like deoxyribozymes (PDz). For example, the PDz may be incorporated to provide an optical read out. See Connelly R P, Verduzco C, Farnell S, Yishay T, Gerasimova Y V. 2019. ACS Chem Biol 14:2701-12. In some embodiments, the active PDz generates a color change due to its catalytic activity. In some embodiments, the color change is from one color to another color (e.g., from green to red). In some embodiments, the color change is from a substantially clear or colorless state to a substantially opaque or colored state (e.g., from clear or colorless to blue). In some embodiments, the color change is from a substantially opaque or colored state to a substantially clear or colorless state (e.g., from blue to clear or colorless). In some embodiments, the color change is a darkening of a color (e.g., from light yellow to dark yellow). In some embodiments, the color change is a lightening of a color (e.g., from dark yellow to light yellow). Any suitable first color and/or second color may be used.

In some embodiments, the PDz activity is that of G-quadruplex (G4) DNA which uses hemin as a cofactor to catalyze peroxidation of colorless organic molecules to produce a colored oxidation product. These substrates include ABTS (2,2′-azino-bis(3-ethylbenzothiazoline-6-sulfonic acid) and TMB (3,3′,5,5′-Tetramethylbenzidine). In some embodiments, the system or methods employ split PDz (sPDz) as split probes for colorimetric detection. In some embodiments two paired portions of a split PDz are brought into proximity of one another in the presence of an analyte to be detected (e.g., a target nucleic acid molecule). In some embodiments, the paired portions are brought together by recruitment of the two portions by partial sequence complementarity with a target nucleic acid molecule. In some embodiments, the paired portions are attached to sequences complementary to sequences of a target nucleic acid molecule, and the uncomplimentary sequences of the paired portions are configured to recombine to form an active PDz. In the absence of the target nucleic acid, the split PDz pair is not brought into proximity with one another and is inactive.

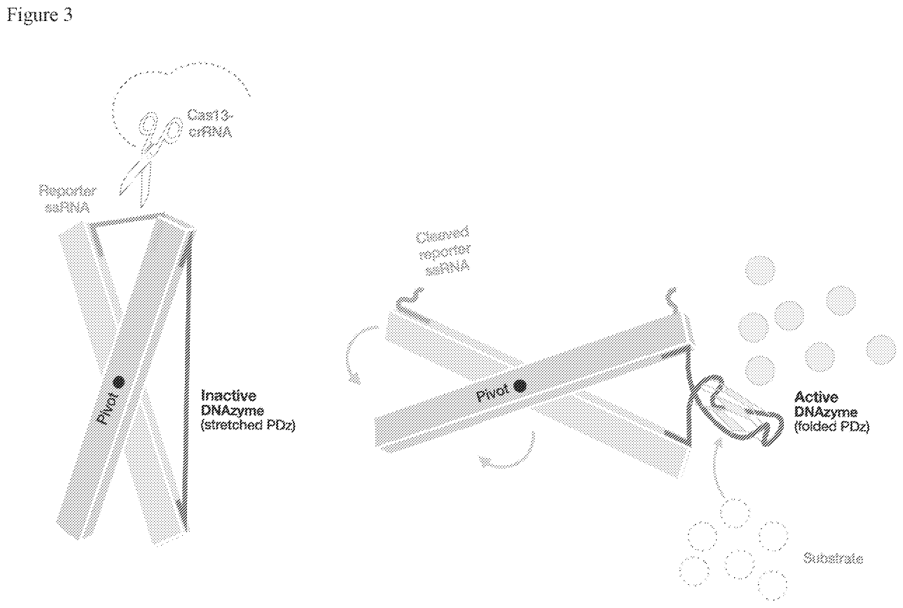

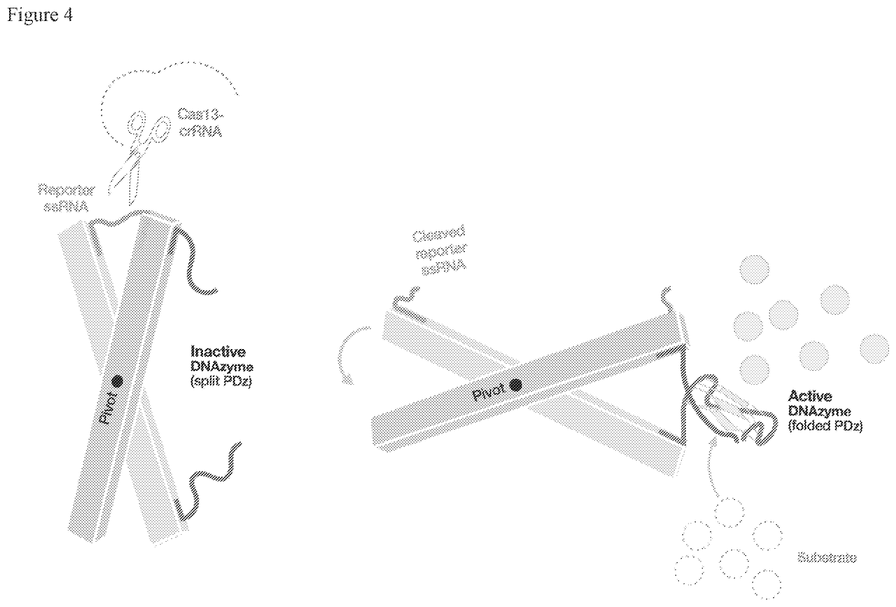

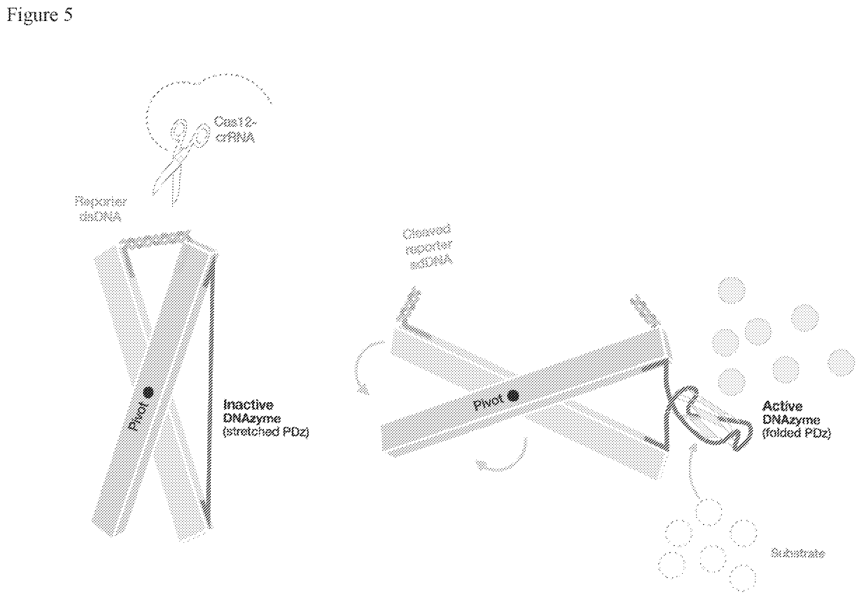

In some embodiments, systems or methods use a hinge-like molecular device that can be prepared in at least two structurally distinct configurations. In one configuration one or more integrated PDzs are rendered inactive (e.g., by separation of a pair of split probes or by holding a PDz in tension). In another configuration, the one or more multiple integrated PDzs are activated (e.g., the pair of split probes are brought into proximity with one another or the PDz is relaxed) (Figures. 3-5). In some embodiments, the inactive PDz is under tension through physically stretching of the nucleic acid strands comprising the PDz sequences (FIGS. 3 and 5). In some embodiments, the inactive PDz is a pair of physically separated nucleic acids wherein each of the pair comprises one portion of a PDz (i.e., an sPDz or split probe) (FIG. 4).

FIG. 3 depicts an exemplary schematic illustration of a switchable hinged-beam DNA nanodevice. The depicted beams are be composed of one or more DNA double-helices connected by a pivot junction. Gerling T, Wagenbauer K F, Neuner A M, Dietz H. 2015. Science 347:1446-52. In the inactive state (left), the nanodevice is constrained by a reporter single-stranded RNA (ssRNA). The reporter ssRNA is flanked by ssRNA segments (anchors) configured to base pair into specific sites on the hinged-beam device (e.g., regions of ssDNA or ssRNA of the beam). In the inactive state the ssDNA containing the PDz sequence is stretched and under tension. A sufficient amount of tension inhibits refolding of the PDz into the active state. The reporter ssRNA may be cleaved by any suitable enzymatic unit, for example, an active Cas13 complexed with an appropriate crRNA. Upon cleavage of the reporter ssRNA, the tension exerted upon the PDz is released, and the PDz can relax. The relaxed PDz to refold into the active PDz state (right).

FIG. 5 depicts an exemplary schematic illustration of a switchable hinged-beam nanodevice similar to that depicted in FIG. 3. In FIG. 5, the inactive state is constrained by a reporter double-strand DNA (dsDNA). The reporter dsDNA is flanked by nucleic acid segments (anchors) (e.g., ssDNA or ssRNA) configured to base pair into specific sites on the hinged-beam device (e.g., regions of ssDNA or ssRNA of the beam). The reporter dsDNA may be cleaved by any suitable enzymatic unit, for example, an active Cas12 complexed with an appropriate crRNA. As in FIG. 3, the PDz is inactive under tension (left), and upon cleavage of the reporter dsDNA, the tension exerted upon the PDz is released, and the PDz can relax. The relaxed PDz to refold into the active PDz state (right).

A ssRNA or dsDNA reporter anchored to the nanodevice may also be used to separate two paired portions of an sPDz (FIG. 4). Upon cleavage of the reporter, the two paired portions of the sPDz can come into proximity with one another and complex to form an active PDz (right).

The active state may be reached by a conformational change that leads to relaxation of the device into another conformation that allows refolding of the active PDz (FIGS. 3 and 5) or recombination of a split PDz (FIG. 4). These refolded or recombined PDzs may then become catalytically active. The state changes can be triggered by cleavage of a reporter ssRNA or a reporter ssDNA or dsDNA. The reporter ssRNA can be firmly anchored in the device by using sufficiently long overhangs. In some embodiments, the system is extended to substantially simultaneous activation of multiple PDzs per cleavage event, by using larger switchable DNA origami bricks. Gerling T, Wagenbauer K F, Neuner A M, Dietz H. 2015. Science 347:1446-52.

In some embodiments, the switchable hinged beam device in which the beams of the device are comprised of multiple DNA helices. In some embodiments, the nanodevice has room for positioning multiple copies of PDz which are each inactive or active according to the conformation of the object. In some embodiments, the nanodevice is constrained in a specific conformational state by a single nucleic acid molecule. For example, in some embodiments, the nanodevice is a static closed variant stabilized by single DNA oligonucleotides. Alternatively, in some embodiments, the nanodevice is a static open variant stabilized by single DNA oligonucleotides.

In some embodiments, the nanodevice is configured to enhance the sensitivity of an associated enzymatic unit (e.g., a Cas13 protein). For example, in some embodiments, the nanodevice is configured such that the limit of detecting target nucleic acids using a PDz remains comparable or even exceeds (e.g., the capacity of detecting even fewer copies of target nucleic acids) that of the conventional, fluorometric assay.

As demonstrated in Example 1, In some embodiments, N=10 activated PDz per cleaved hinge device may be used, such that t_{READ} will be around 40 minutes. This time is directly comparable to the performance of the conventional, fluorescence-based Cas13 assay. Fozouni P, Son S, Diaz de Leon Derby M, Knott G J, Gray C N, et al. 2020. Cell. In some embodiments, N=50 may be used, the time may be cut to less than 20 minutes, and the colorimetric assay may actually outperform its fluorometric basis. Fozouni P, Son S, Diaz de Leon Derby M, Knott G J, Gray C N, et al. 2020.

t_{READ(N=10)}=38 mins

t_{READ(N═50)}=17 mins

t_{READ(N=500)}=5 mins

In some embodiments, the substrate ssRNA is in large excess over Cas13 and is cleaved by Cas13. Any suitable concentrations of cleaving protein (e.g., Cas13) may be used. Suitable concentrations may be in the range of 100 nM, 200 nM, 300 nM, 400 nM, 500 nM, 600 nM, 700 nM, 800 nM, 900 nM, or 1 mM. In a preferred embodiment, Cas13 is present in the range of about 400 nM. In some embodiments the concentration of a PDz substrate (e.g., ABTS) is present in a concentration similar to that of the catalytic converter device. In some embodiments, the substrate is present at about 100 nM, 250 nM, 500 nM, 600 nM, 700 nM, 800 nM, 900 nM, 1 mM, 1.25 mM, 1.5 mM, 2 mM, 2.5 mM, 3 mM, 4 mM, 5 mM, 6 mM, 7 mM, 7.5 mM, 8 mM, 9 mM, 10 mM, 20 mM, 25 mM, 30 mM, 40 mM, 50 mM, 60 mM, 70 mM, 75 mM, 80 mM, 90 mM, 100 mM, 200 mM, 250 mM, 300 mM, 400 mM, 500 mM, 600 mM, 700 mM, 750 mM, 800 mM, 900 mM, or 1000 mM. In preferred embodiments, the substrate is present in the mM range. In preferred embodiments the substrate is present at about 1 mM-10 mM.

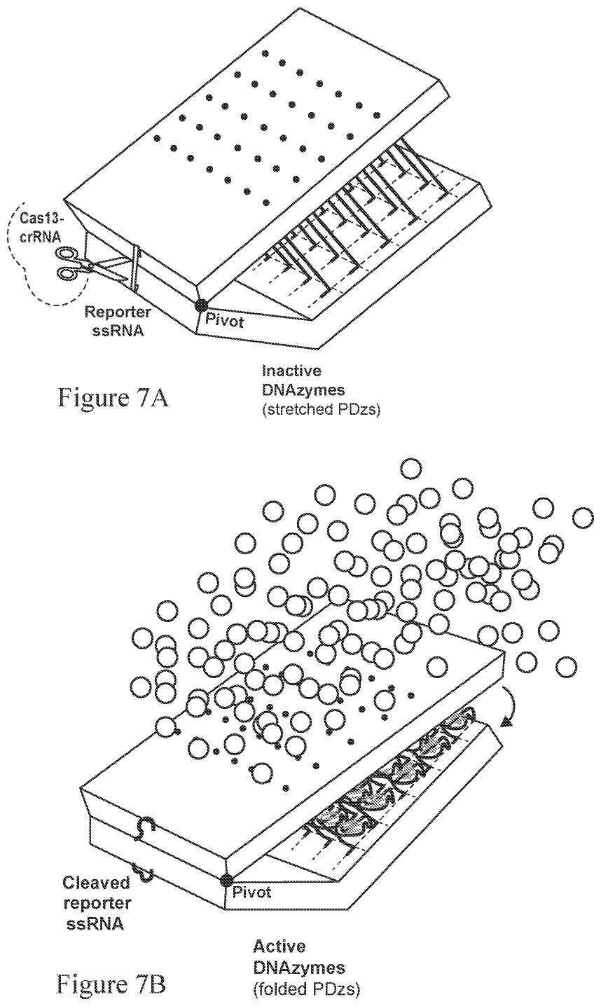

In some embodiments, the PDz motifs may be held in present in stretched or split states as part of a clamshell device (FIGS. 7A-7B). In some embodiments, the inactive state of the clamshell can be converted into an active state by a single reporter ssRNA cleavage event.

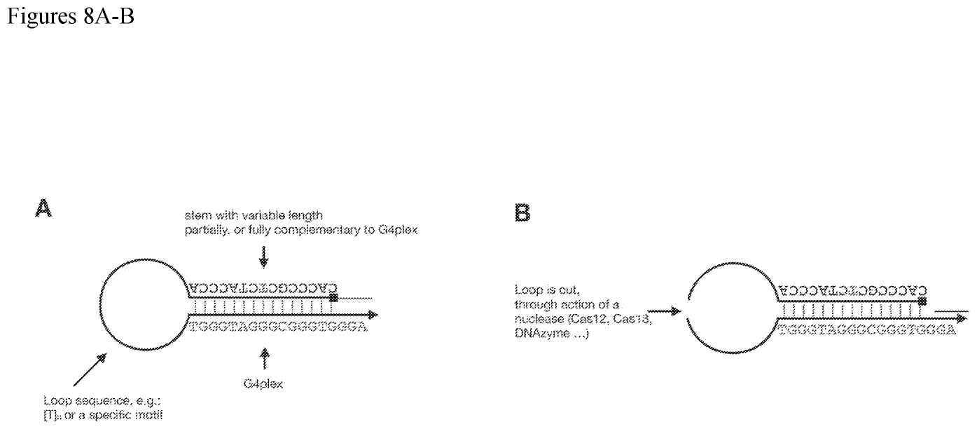

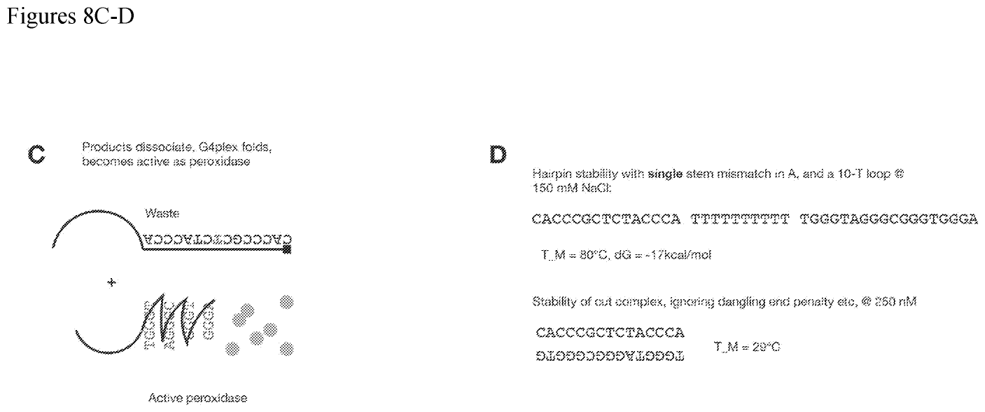

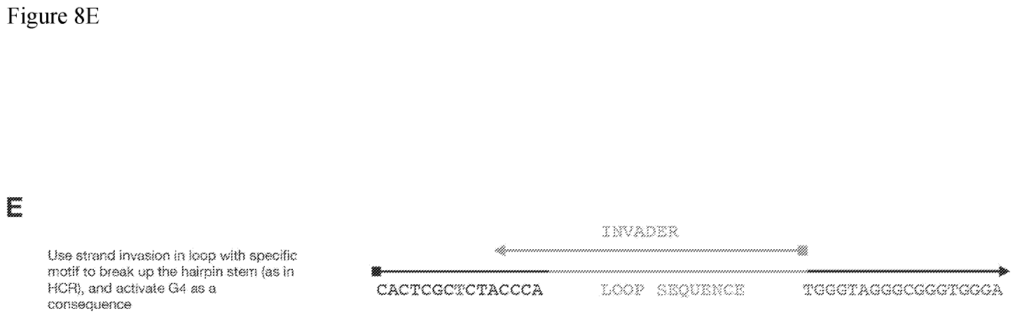

In some embodiments, the systems or methods employ a single nucleic acid strand comprising a PDz sequence (FIG. 8A). In some embodiments, the PDz's catalytic activity is inhibited by a partially self-complementary region of the strand (FIG. 8A). In some embodiments, the strand also includes a hairpin loop region that is composed of RNA or DNA nucleotide bases that may be targeted by an enzymatic cleaving unit (e.g., a CRISPR-Cas13 or -Cas12 system) (FIG. 8B). In some embodiments, the cleavage of the hairpin loop permits is triggered by activation of the enzymatic unit (e.g., an amplifier output). The cleavage permits dissociation of the PDz sequence from the complementary sequence, allowing the PDz to refold into a catalytically active state (FIGS. 8C-8D). In some embodiments dissociation is encouraged by the self-complementary region not being fully self-complementary. For example, the self-complementary region may possess one or more base pair mismatches or abasic sites (FIGS. 8A-8C). In some embodiments, the active PDz is configured to produce a colorimetric output visible with the unaided eye (FIG. 8C).

In some embodiments, the amplifier output may be in the form of a nucleic acid that is complementary or semi-complementary to a portion of the hairpin loop (e.g., an invader strand).

In such embodiments, the system or method does not require an enzymatic unit (e.g., an active CRISPR-Cas complex). In some embodiments the invader strand is a single stranded nucleic acid that binds to the hairpin loop promoting dissociation of the PDz sequence and the self-complementary sequence (FIG. 8E). In some embodiments, the dissociation is similar to hybridization chain reaction systems.

In some embodiments, the detection of different analytes provides the same detectable signal (e.g., the same colorimetric signal). In some embodiments, a positive detection of the molecule is determined by all sensors (e.g., reservoirs) providing a positive signal.

In some embodiments, the detection of analytes provides a different detectable signal (e.g., detection of a first analyte provides a first colorimetric signal and detection of a second analyte provides a second colorimetric signal). In some embodiments, the different detectable signals contrast with one another. In some embodiments, the detection using different detectable signals is provided for in the same reservoir. In some embodiments, detection of the target molecule is determined by a composite signal.

In some embodiments, the detection module is the third layer of a multilayer architecture.

In some embodiments, the detector layer comprises an activatable (e.g., gated) peroxidase. In some embodiments, the peroxidase is gated and is configured to become an active peroxidase upon cleavage of the gating mechanism. In some embodiments, an active peroxidase reacts enzymatically with one or more substrates to provide one or more products. In some embodiments, the peroxidase is a G-quadraplex peroxidase (G4). In some embodiments, the peroxidase of the system reacts with ABTS or TMB to create a color change visible to the naked eye.

In some embodiments, the third layer is the detection module comprising one or more caged nucleic acid peroxidase enzyme. In some embodiments, the one or more caged nucleic acid peroxidase enzyme is capable of catalyzing the conversion of an initially colorless compound into a colored compound to produce a naked-eye readable output signal once activated.

In some embodiments, the caged enzyme peroxidase can be activated only through amplified output strands generated in the second layer. In this case, the N output strands from the second layer generated per trigger molecule generate N active enzyme peroxidases. In some embodiments, the number of colored compound molecules produced in the third layer grows linearly in time with N*k_{cat}*t, where k_{cat} is the catalysis rate of the active peroxidase. In some embodiments, a split G-quadruplex DNAzyme probe is used. In some embodiments, a nucleic acid motif recruits the two split pieces of the probe, thereby recombining the active G quadruplex. Connelly R P, Verduzco C, Farnell S, Yishay T, Gerasimova Y V. 2019. ACS Chem Biol 14:2701-12

In some embodiments, the caged enzyme peroxidase can be uncaged by the activity of Cas proteins activated in the second layer. Over time, Cas proteins will activate more and more peroxidases. If M active Cas molecules have been produced in the second layer, the third layer is capable of generating M*k_{cat}*k_{cat}*t₂ colored molecules per trigger molecule, provided that sufficient reactants (e.g., substrate and reaction co-factors) are present. In some embodiments, the second layer uses a nucleic-acid cascade and Cas proteins in series. N*M active Cas molecules per trigger molecule will be generated. The third layer is capable to then generate N*M*k_{cat}*k_{cas}*t₂ colored molecules per trigger molecule, provided that sufficient reactants (e.g., substrate and reaction co-factors) are present. In some embodiments, a G-quadruplex sequence motif sequestered in a DNA hairpin stem is used. By hiding the functional G-quadruplex sequence in the hairpin it cannot function as a peroxidase. The G-quadruplex enzyme motif is therefore inhibited as long as it stays buried in the hairpin loop. The loop in the hairpin can be RNA or DNA. In some embodiments, trigger-strand activated Cas13 or Cas12a proteins from the second layer can digest the loop. In some embodiments, the hairpin stem strands will dissociate, and the G-quadruplex sequence will be able to fold into the active G-quadruplex structure. In some embodiments, hybridization of DNA strand motifs created by protein-free paths in the second layer invade the hairpin loop or regions of the hairpin stem such that the G quadruplex motif can refold and become active.

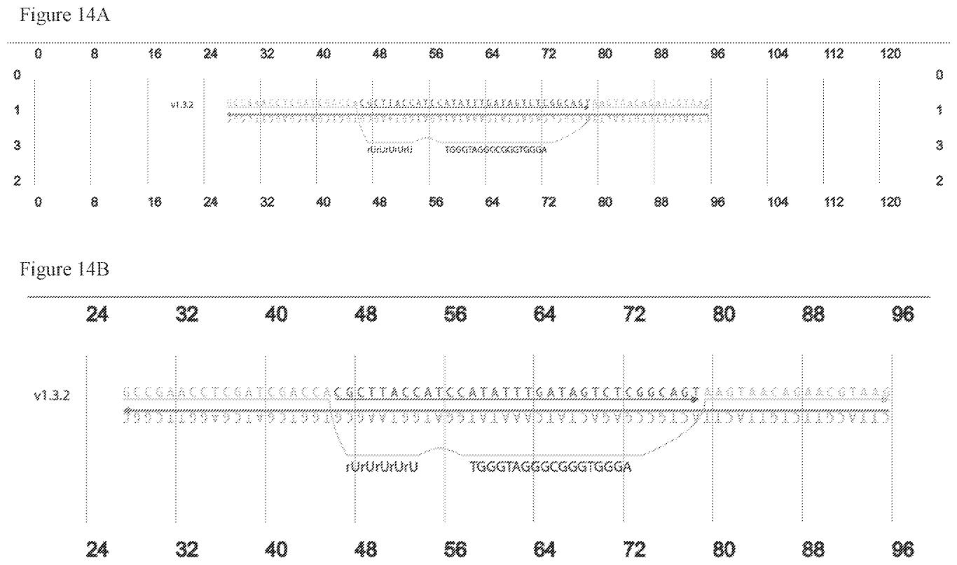

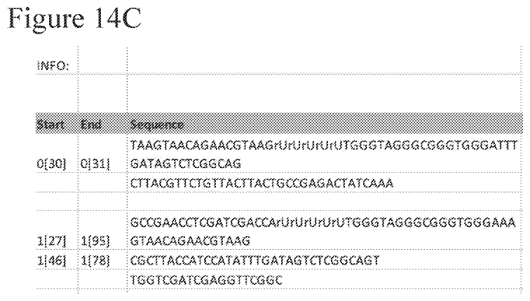

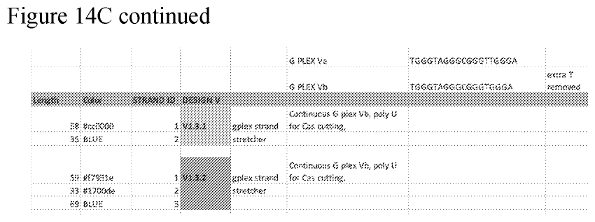

In some embodiments, the caged enzyme peroxidase can be activated either through amplified output strands from the second layer via strand-displacement reactions or by ssRNA or ssDNA degrading activity from activated Cas proteins produced in the second layer. The third layer will be capable of generating N*k_{cat}*t+M*k_{cat}*k_{cas}*t₂ colored molecules per trigger molecule. In some embodiments, a G-quadruplex sequence motif is stretched out on a DNA duplex rack, which inhibits formation of the active G-quadruplex structure. For example, see design v1.3.2 at Example 3 (FIG. 14A-14C). The peroxidase is inhibited as long as it stays stretched on the DNA duplex rack. In some embodiments, the stretched-out G-plex can be flanked with a single-stranded (e.g., UUUUU) region. In some embodiments, the trigger-strand activated Cas13 or Cas12a proteins from the second layer can cut the single-strand (e.g., UUUUU). The G-plex, when not under tension, is able to relax and fold into the active G-quadruplex structure. In some embodiments, hybridization of DNA strand motifs created by protein-free paths in the second layer strand-displace the stretched-out G4 from the DNA rack such that the G quadruplex motif is able to fold and become active.

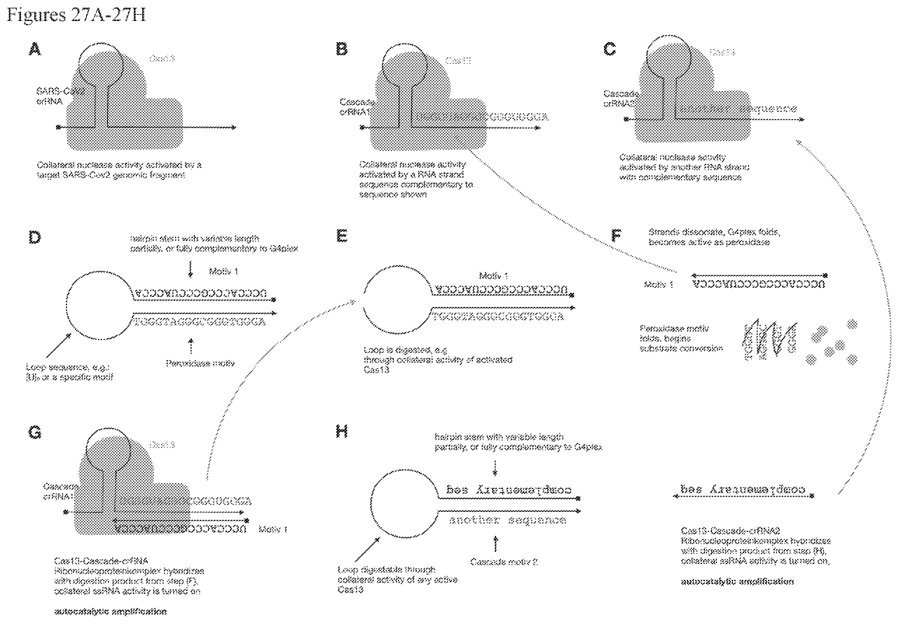

In some embodiments, the modules are configured to provide an auto-catalytic cascade to amplifying the signal from a target. In some embodiments, the system or method employs one or more of the following steps:
introduction of a first inhibited nuclease (e.g., an activatable Cas protein (e.g., Cas12 or Cas13) or an activatable Cas protein complexed with CRISPR) and introduction of a first activating sequence (e.g., a polynucleotide released as a translator output) to yield a first active nuclease (e.g., an active Cas proteins (e.g., Cas12 or Cas13) or an active Cas protein complexed with CRISPR);
introduction of a first active nuclease (e.g., an active Cas protein (e.g., Cas12 or Cas13) or an active Cas protein complexed with CRISPR) and introduction of an inhibited colorimetric actuator (e.g., an activatable nucleic acid peroxidase, substrates, and cofactors) to yield an active colorimetric actuator (e.g., an active nucleic acid peroxidase, substrates, and cofactors);
introduction of a first active nuclease (e.g., an active Cas proteins (e.g., Cas12 or Cas13) or an active Cas protein complexed with CRISPR) and introduction of a second masked activating sequence (e.g., an activatable element) to yield a second activating sequence (e.g., an active element);
introduction of a second inhibited nuclease (e.g., an activatable Cas protein (e.g., Cas12 or Cas13) or an activatable Cas protein complexed with CRISPR) and introduction of a second activating sequence (e.g., an activatable element) to yield a second active nuclease (e.g., an active Cas protein (e.g., Cas12 or Cas13) or an active Cas protein complexed with CRISPR); and
introduction of a second active nuclease (e.g., an active Cas protein (e.g., Cas12 or Cas13) or an active Cas protein complexed with CRISPR) and introduction of an inhibited colorimetric actuator (e.g., an activatable nucleic acid peroxidase, substrates, and cofactors) to yield an active colorimetric actuator (e.g., an active nucleic acid peroxidase, substrates, and cofactors) (FIG. 27).

In some embodiments, the Cas protein is a Cas12 protein, Cas13 protein, subvariant protein thereof. In some preferred embodiments, the Cas protein is a Cas12a protein. In some embodiments, the system or method provides programmatic activation of one or more RNAse proteins in the presence of one or more activating sequences.

In some embodiments, amplifying hairpin probes (FIGS. 27D and 27H) may be provided in high concentrations. In some embodiments, amplifying hairpin probes are provided in the hundreds of nanomolar range. In some embodiments, the amplifying hairpin probes are provided at about 100 nM or greater, about 200 nM or greater, about 300 nM or greater, about 400 nM or greater, about 500 nM or greater, about 600 nM or greater, about 700 nM or greater, about 800 nM or greater, about 900 nM or greater, about 1,000 nM or greater.

### General Architecture

In some embodiments, the architecture is a layered (tiered) system for detecting nucleic acids comprising a translation layer, an amplification layer, and a detection layer (FIG. 18). In some embodiments, the translation layer receives a plurality of nucleic acid input signals and converts them into a single signal (e.g., the translator output). The nucleic acid input signals may include ssRNA or ssDNA. The translator output may include ssDNA, ssRNA, dsDNA, dsRNA, and/or a partially duplexed dsDNA, dsRNA, or RNA/DNA hybrid. In some embodiments, the amplification layer amplifies the translator output and creates a second signal (e.g., the amplifier output). In some embodiments, the detection layer converts the amplifier output into an optical output (e.g., a colorimetric change).

In some embodiments, the three layers of the detection system are incorporated into a lateral flow assay. In some embodiments, the three layers of the detection system are incorporated into a paper strip assay. In some embodiments, the detection system is incorporated by means of gel, polymer, desiccation, lyophilization or some combination thereof the lateral flow assay. In some embodiments, the incorporation is achieved by means of soaking, impression printing, inkjet printing, or some combination thereof. In some embodiments, the substrate of incorporation is a polymer film, nonwoven, paper, or fiber glass. In some embodiments, the incorporation results in immobilization of the detector layer at a specified immobilization site. In some embodiments, the immobilization of the detector layer is reversible upon release by the amplifier output and is carried away through diffusion or capillary action into a runoff site. In some embodiments, the location of the optical output designates the presence of the measured nucleic acid wherein a negative result is designated by an optical output at the original immobilization site and a positive result is designated by an optical output in the runoff site. In some embodiments, the immobilization of the detector layer is reversible upon release by diffusion or capillary action. In some embodiments, the amplifier output destroys the detector layer wherein a negative result is designated by an optical output and a positive result is designated by the absence of an optical output. In some embodiments, the immobilization of the detector layer is irreversible. In some embodiments, a negative result is designated by the absence of an optical output and a positive result is designated by an optical output.

In some embodiments, the system or method employ a solid state system. In some embodiments, the system or methods may incorporate agarose gels, polyacrylamide gels, and other similar materials. These gels may be cured into a well, extruded into film, soaked into absorbent material (e.g., nonwoven, cloth, fibers), or prepared in another appropriate manner. In some embodiments, the system or method may incorporate surface immobilization, including but not limited to, nanoparticles and acrylamide copolymerization. In some embodiments, the system or method may incorporate techniques, including but not limited to, physical adsorption, covalent attachment, bioaffinity immobilization of some nucleic acids, LFA, and surface modified chips.

How molecules are immobilized may affect how the sensor functions. For example, affixing RNA molecules tethered to a G4 on one end and the surface on the other, may permit Cas to cut the tether as the sample flows. In some embodiments, the G4s will be carried to where the hemin, ABTS, peroxide is and react. This can result in a localized color change at the end of the strip if positive. If diffusion is an issue, a color change may still occur in the middle which could signify a negative result

In some embodiments, the readout results from where the color change occurs. For example, a positive result may be indicated by a color change near the end of the form and a negative result may be indicated by a color change near the middle of the form. In some embodiments, an RNA based G4 may be tethered in near the middle of the form, and the ABTS, Hemin, and peroxide is provided the end of the form. In some embodiments, an activated Cas protein digests the G4, preventing a color. In some embodiments, an inactive Cas protein does not digest the G4, and a color occurs near the middle of the strip.

In some embodiments, the system may incorporate powder, for example, lyophilized components. These may be used for surface immobilization. In some embodiments wherein the system includes a strip test without the need to immobilize, the powder may be added to an adhesive attached to some absorbent material or pad which carries the sample past the powder. If all systems can be rehydrated into an active state, the sample is capable of activating the powdered components. In some embodiments, a coating powder may be incorporated onto nonwoven forms.

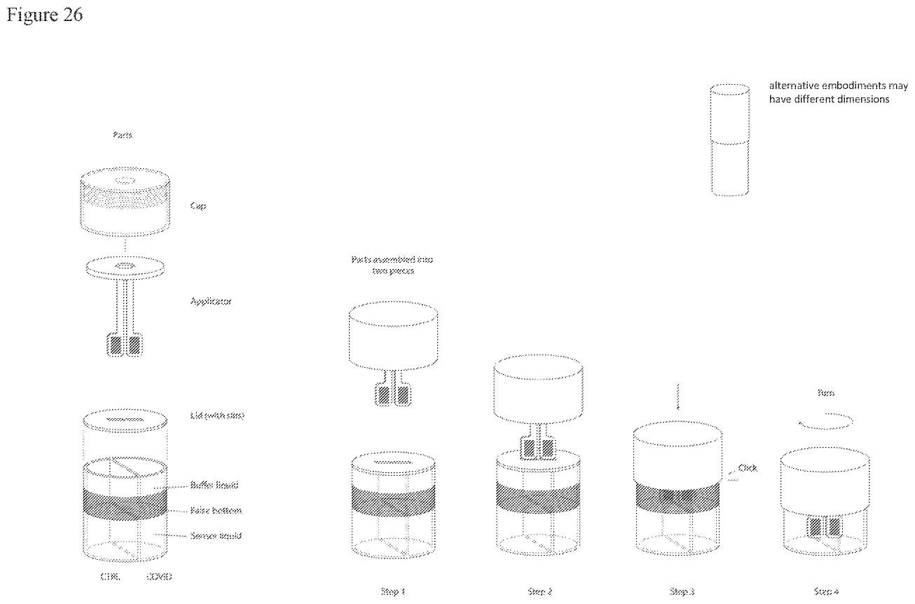

In some embodiments, the system or method employ a non-solid state system, including, for example, in-solution system (FIG. 26).

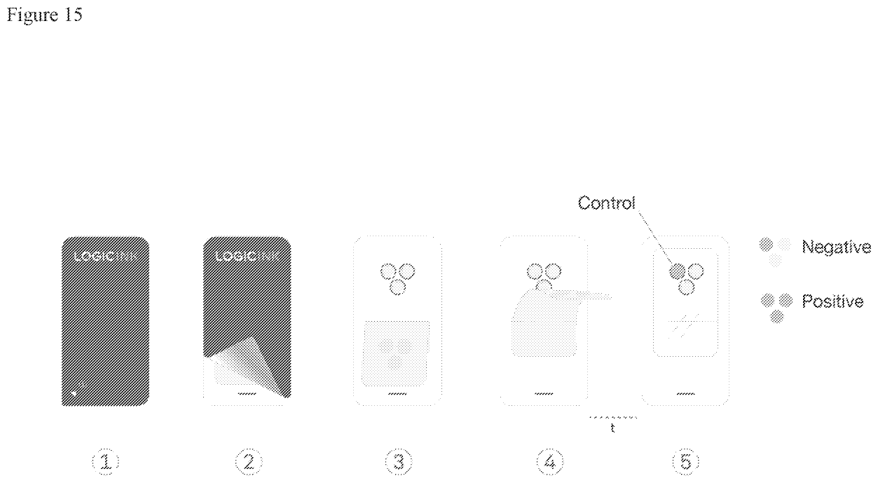

In some embodiments, the system or method provides for detection of multiple analytes. In some embodiments, the detection of multiple analytes is provided separately in a single form-factor (e.g., in separate reservoirs) (FIGS. 15-18). In some embodiments, the detection of multiple analytes is provided in together in a single form-factor (e.g., in a single reservoir).

In some embodiments, the second analyte is of a the same or a similar molecular type as the first analyte. For instance, in embodiments where the first analyte is a nucleic acid (e.g., a DNA or an RNA), the second analyte may also be a nucleic acid (e.g., a second DNA or RNA). As a further example, the first analyte may be a virus protein and the second analyte may be antibodies associated with infection by the virus. In some embodiments, the first and second analyte may be the same analyte (e.g., the same DNA consisting of the same DNA sequence). In such embodiments, the system or method may provide for redundant detection of the same analyte by separate sensors. It will be readily understood that such redundant, independent detection of the same analyte provides an increased confidence in detections of the analyte. In some embodiments, the second analyte is of a different molecular type as the first analyte. For instance, in embodiments where the first analyte is a nucleic acid, the second analyte may be a protein, polypeptide, or an oligonucleotide.

In some embodiments, the system or method detects an analyte associated with an infectious agent (e.g., a virus) and an analyte associated with an immune response to the infectious agent (e.g., an antibody). It will be understood that the detecting the combination of an infectious agent analyte and an immune response analyte may be of critical importance in monitoring how quickly and where a vector-borne disease is spreading and correlating such information with the rate at which a population is able to become immune against it. While previously available detection systems (e.g., antibody detections) permit reactive responses to infectious agents, the instant system or method provides an advantage in providing proactive approaches to dealing with infectious diseases by providing both immune response detection and infectious agent detection (e.g., detection of a virus itself from a person's saliva, an object's surfaces, and/or the environment)

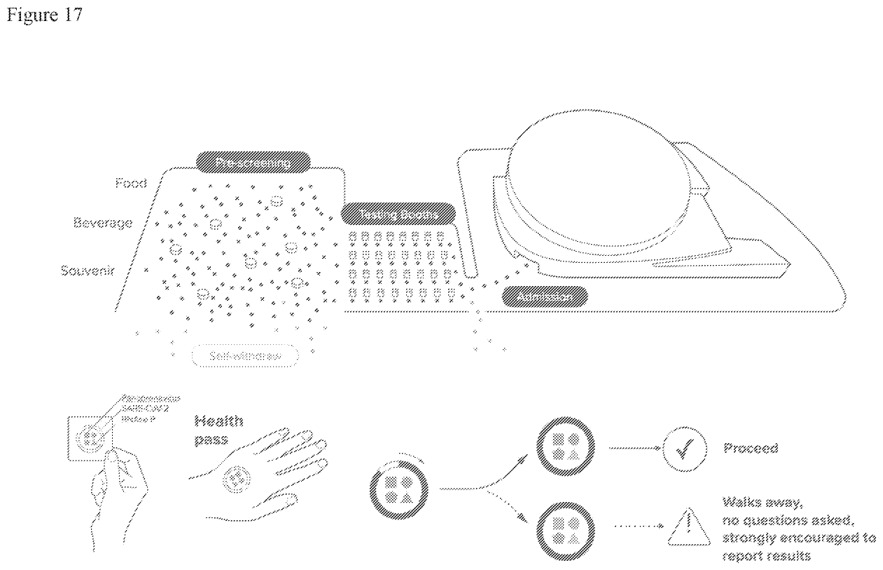

Any of the designs or methods disclosed herein may be in any appropriate form-factor or use-case including, but not limited to, a skin wearable, a sticker on an object's surface, a two-dimensional applique, or a substrate (e.g., a polymeric film substrate). In some embodiments, the form-factor or use-case may further define one or more reservoir protected by a porous membrane. A reservoir may contain a biosensor system focused on a one or more specific analytes, for example a specific RNA or DNA sequence being targeted. For example, FIG. 17 depicts an exemplary form-factor comprising four separate reservoirs. In FIG. 17, the four reservoirs correspond to a biosensor system for detecting an analyte associated with SARS-CoV-2 (i.e., SARS-CoV-2), an analyte associated with viruses from the CoV family (pancoronavirus), an additional analyte target (RNAseP), and a control target. In some embodiments the reservoirs act as homogeneous assays where all biosensor reactions occur concurrently. The reservoirs provide separation among the biosensor systems to avoid cross-reaction effects.

The reservoir may be protected with a lid or seal that can be peeled or partially peeled back. In some embodiments, the lid or seal is replaced after a source (e.g., saliva deposited by a lick) is provided. The form-factor may comprise a control on the borders of one or more of the reservoirs that provides an indication upon the application of a source. For example, the border may change color (e.g., from clear to blue) when presented with a detectable characteristic of the source (e.g., a pH associated with a source such as saliva). Preferably, the indication is instantaneous or near-instantaneous. The indication corresponds to a sufficient amount of source provided to the associated reservoir.

In some embodiments, the performance of the system or method may be modified by any suitable means. Without limitation, such modifications include concentrating a sample (e.g., saliva) in a solid state collector by spatially constraining it to some active area, activating multiple peroxidases (e.g., G4) per amplifier output (e.g., active Cas proteins such as Cas 12 and/or Cas13) cutting event, increasing the rate of substrate to product turn over (e.g., peroxidation) by using more substrate (e.g., ABTS and H₂O₂), and reducing residual background activity of non-switchable (e.g., consistently active) peroxidases by, for example, purification.

In some embodiments, the chemistry of the sensor can be calibrated to be less or more sensitive to different concentrations of the target analyte. In some embodiments, the system includes one, two, or more levels of quantification (e.g., energy bars). These levels of quantification may be used to finetune certain variables (e.g., the number of PDzs).

### EXAMPLES

### Example 1

### Kinetic Demonstration of Catalytic Conversion of PDz Nanodevice

Provided here is modeling of the power of exemplary designs of the system or methods.

The kinetical analysis of the nanodevice demonstrates its power of the catalytic conversion. This analysis assumes that both Cas13 and the PDz operate with Michaelis-Menten kinetics in the limit of large substrate excess, having specific catalytic rates. For Cas13, k_{cat,Cas13}=600 s⁻¹ (2). The G-quadruplex-based PDzs typically have slower catalytic rates, on the order of k_{cat,PDz}=1 s⁻¹. Li W, Li Y, Liu Z, Lin B, Yi H, et al. 2016. Nucleic acids research 44:7373-84.

In the limit of substrate excess, substrate turnover by the target-RNA activated Cas13 will occur with a constant rate. Therefore, the concentration of cleaved, and thus activated hinge devices changes as follows:

$$\frac{d}{dt} {C}_{cleaved nanodevice} = {k}_{cat , Cas 13} \cdot {C}_{active cas 13} ⇒ {C}_{cleaved nandevice} ( t ) = {k}_{cat , Cas 13} \cdot {C}_{active cas 13} \cdot t . \qquad ( equation 1 )$$

The concentration of C_{active cas13} active Cas13 may be assumed to be identical to the concentration of the target RNA. Hence, C_{active cas13} is replaced with C_{target RNA}.

The turnover of oxidized (colored) ABTS by the cleaved hinged device will in turn be given by

$$\frac{d}{dt} {C}_{ABTS} = {k}_{cat , PDz} \cdot N \cdot {C}_{cleaved nanodevice} ( t ) = {k}_{cat , PDz} \cdot {k}_{cat , Cas 13} \cdot N \cdot {C}_{target RNA} \cdot t$$

Here, equation 1 has already been used and N has been introduced to denote the number of PDz that get activated per cleaved nanodevice. As a result, the following is obtained:

C_{ABTS}(t)=½·k_{cat,PDz}·k_{cat,Cas13}·N·C_{target RNA}·t² (equation 2).

Equation 2 is rearranged to compute the time needed t_{READ} for having reached a particular target concentration C_{READ} of converted, and thus colored ABTS:

$${t}_{READ} = \sqrt{\frac{2}{N} \cdot \frac{1}{{k}_{cat , PDz} \cdot {k}_{cat , Cas 13}}} . \qquad ( equation 3 )$$

Using the numbers for the rates as given above, 30 fmol/L for C_{target RNA} (which corresponds to approximately 100 copies per microliter, and 0.5 mmol/l for C_{READ}, the following is arrive at:

$${t}_{READ} ( N ) = \sqrt{\frac{2}{N} \cdot \frac{1 \cdot {10}^{- 3} mol / l}{3 \cdot {10}^{- 4} mol / l} \cdot \frac{1}{1 {s}^{- 1} \cdot 600 {s}^{- 1}}} \approx \sqrt{\frac{2}{N}} \cdot 87 mins$$

### Example 2

### System Performance

Provided here is an illustration of the performance of an exemplary embodiment of the system or method comprising three layers.

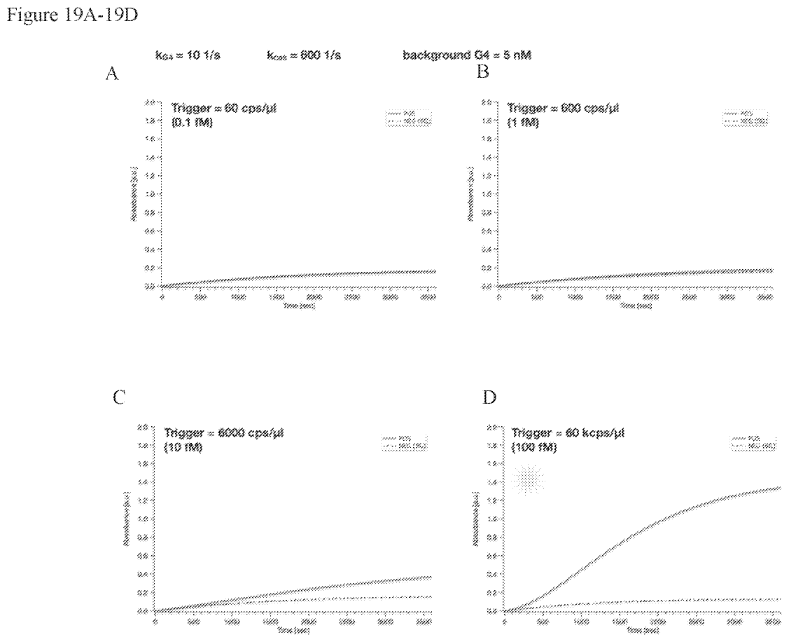

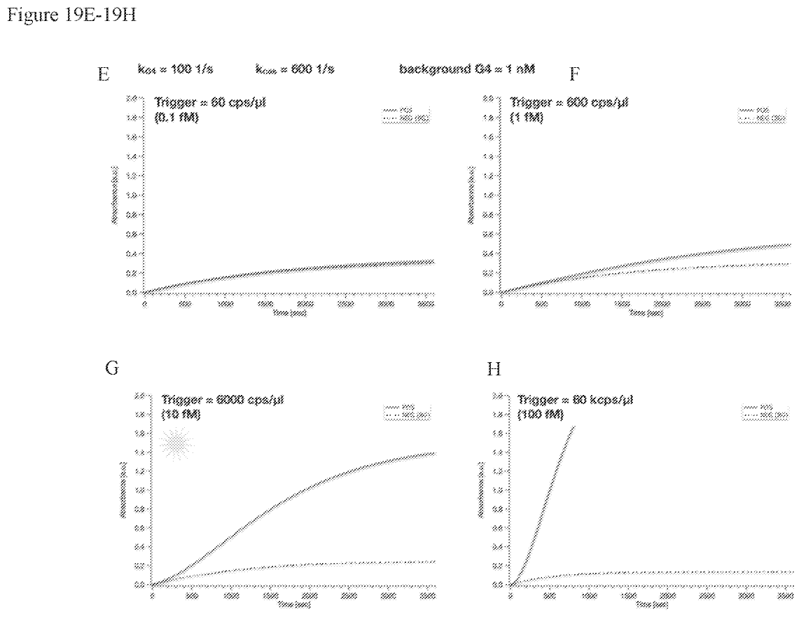

FIGS. 19A-19L illustrate the performance of the combined action of the second layer (e.g., amplification module) and the third layer (e.g., detection module), as calculated with a simple numerical model. The model computes the concentration of catalytically active G quadruplex peroxidases (C_{G40N}), the concentration of transparent molecules (C_{TRANS}) (e.g., a substrate) that are turned over by the peroxidase into “blue” molecules (C_{BLUE}) (e.g., a product). Active peroxidases are being generated by active Cas proteins (e.g., Cas13 or Cas12) with a rate that depends on the initial concentration of active Cas proteins and the catalysis rate k_{Cas}. The initial concentration of active Cas proteins is assumed to be identical to the concentration of trigger strands (e.g., the translator output) produced by the first layer (e.g., translator module) or by an amplifying cascade provided by the second layer. The rate k_{G4} at which G quadruplex peroxidases turnover the substrate molecule into the product varies with time. k_{G4} depends on the current concentration of hydrogen peroxide and the concentration of available substrate molecules.

$$\frac{d}{dt} {C}_{G 4 - ON} ( t ) = + {k}_{CAS} \cdot {C}_{CAS - ON} ( t ) \\ \frac{d}{dt} {C}_{BLUE} ( t ) = + {k}_{G 3} ( t ) \cdot {C}_{G 4 - ON} ( t ) \\ \frac{d}{dt} {C}_{{H}_{2} {O}_{2}} ( t ) = - {k}_{perox} \cdot {C}_{{H}_{2} {O}_{2}} ( t ) - {k}_{G 4} ( t ) \cdot {C}_{G 4 - ON} ( t ) \\ {C}_{TRANS} ( t ) = {C}_{TTRANS} ( {t}_{0} ) - {C}_{BLUE} ( t ) \\ \frac{d}{dt} {C}_{BLUE - BG} ( t ) = + {k}_{G 4 , BG} ( t ) \cdot {C}_{G 4 - ON} ( {t}_{0} ) \\ {k}_{G 4} ( t ) = {k}_{G 4}^{0} \cdot ( \frac{{C}_{{H}_{2} {O}_{2}} ( t )}{{C}_{{H}_{2} {O}_{2}} ( {t}_{0} )} ) \cdot ( \frac{{C}_{TRANS} ( t )}{{C}_{TRANS} ( {t}_{0} )} )$$

FIGS. 19A-19L depict a simplified numerical model for computing the performance of the double-catalytic system consisting of activated Cas proteins (e.g., Cas12 or Cas13), which, in turn, activate accumulating G4 quadruplex peroxidases. C_{BLUE-BG} denotes a negative control reaction run in parallel with residual initially active G quadruplex peroxidases. The rate of substrate turnover, k_{G4, BG}, is analogous to k_{G4}.

Solid lines depict the predicted performance of the combination of the second and third layers (e.g., the amplification and detection modules), for different concentrations of input trigger strands (e.g., translator output), and different concentrations of reactants. Dashed lines depict negative controls. k_{G4} can be scaled with the overall hydrogen peroxide concentration. Stars (FIGS. 19D, 19G, and 19I) indicate where the limit of detection can be reasonably be expected, based on a per-eye discrimination of negative control (background) versus actual test sample.

k_{G4} is modeled at 10 per second (FIGS. 19A-19D), 100 per second (FIGS. 19E-19H), and 100 per second (FIGS. 19I-19-L). k_{Cas} is modeled at 600 per second (FIGS. 19A-19H) and 1200 per second (FIGS. 19I-19-L). Background active peroxidase (G4) is modeled at 5 nM (FIGS. 19A-19H) and 1 nM (FIGS. 19I-19-L). Trigger (e.g., translator output) concentration is modeled at 60 cps/μl (0.1 fM) (FIGS. 19A, 19E, and 19I), 600 cps/μl (1 fM) (FIGS. 19B, 19F, and 19J), 6,000 cps/μl (10 fM) (FIGS. 19C, 19G, and 19K), and 60,000 cps/μl (100 fM) (FIGS. 19D, 19H, and 19L).

Assuming an amplification level N=10 for the first layer (e.g., the translator module), the expected limit of detection corresponds to a target strand concentration of approximately 0.5 fmol/L (300 cps/μl). If N=1, the limit of detection is expected to correspond to a target strand concentration of approximately 5 fmol/L.

### Example 3

### Activatable Peroxidase Designs

Provided here is the performance of exemplary design variants of activatable peroxidases that can be used with the system or methods.

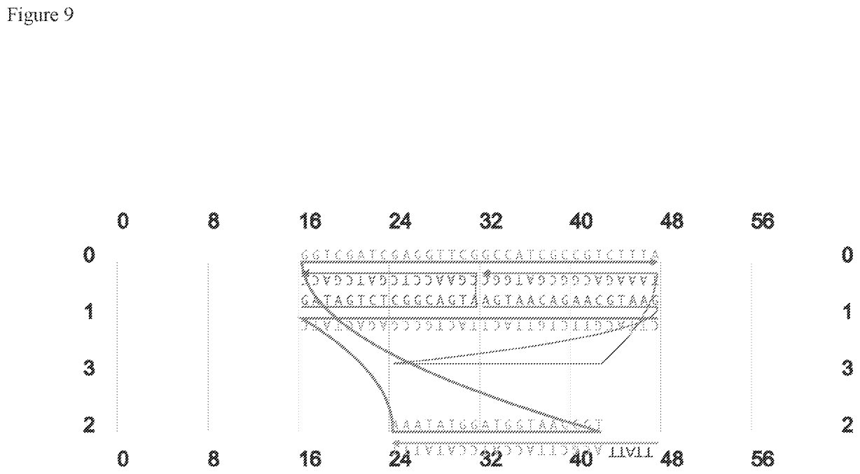

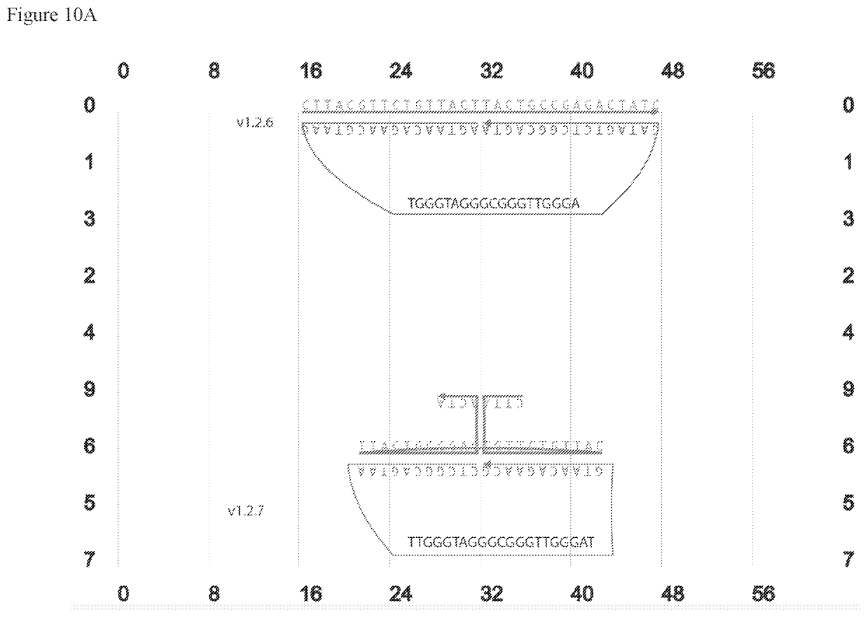

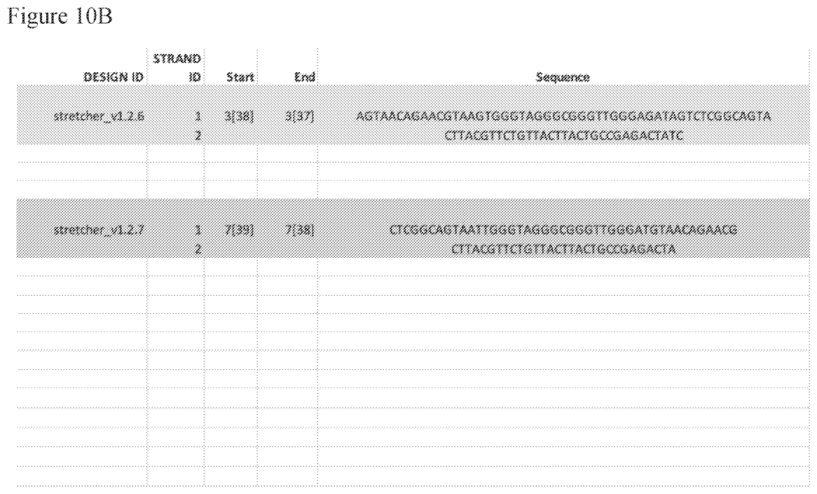

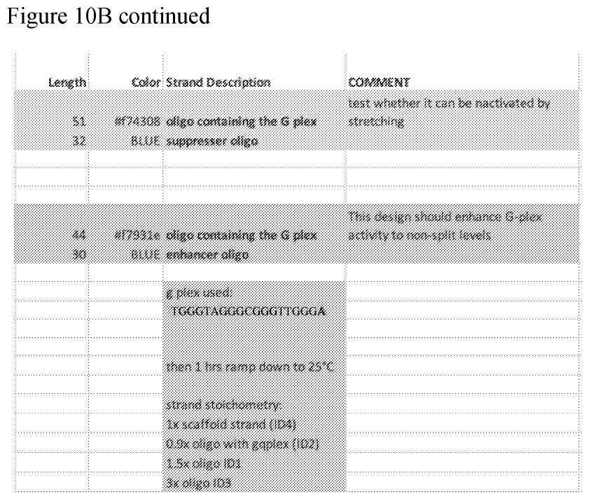

Exemplary sequence designs include v1.2 G-plex (PDz) stretcher (FIG. 9). v1.2.6 and v1.2.7 are examples of two designs for deactivation and activation of circularized G-plex probes. Both are circularized G-plex (PDz) variants containing the G plex suppressor oligo (FIGS. 10A-10B).

v1.2.8 to v1.2.11 (with subvariants a and b) represent a total of 8 designs covering split and continuous G plex variants (in two sequence modifications). They present two slightly different ways of constraining the ends of the activatable G plex.

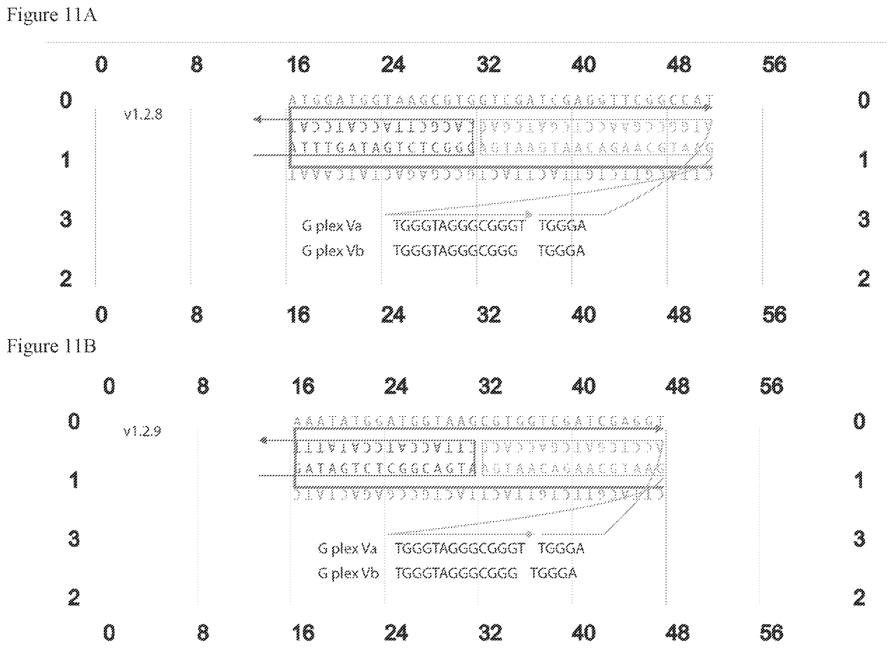

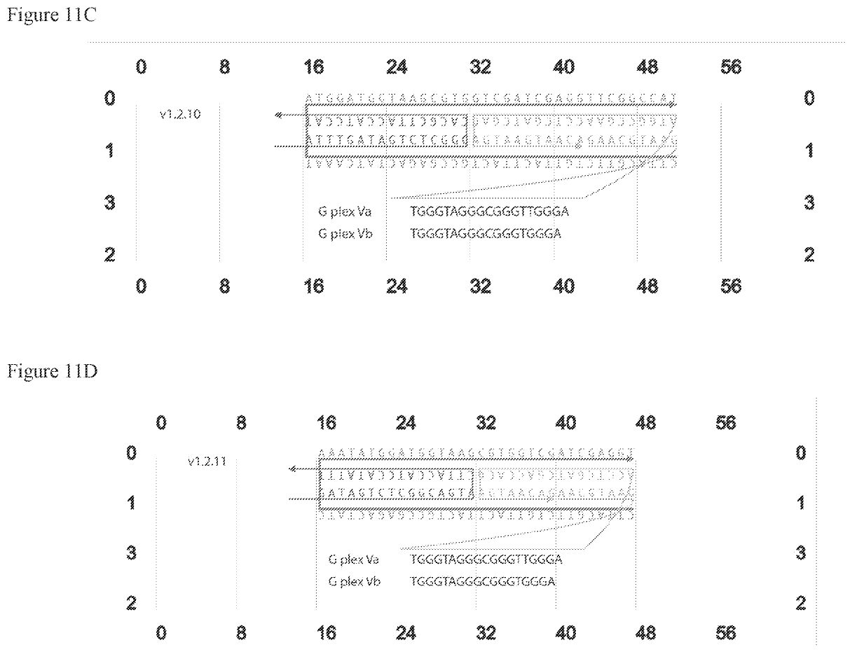

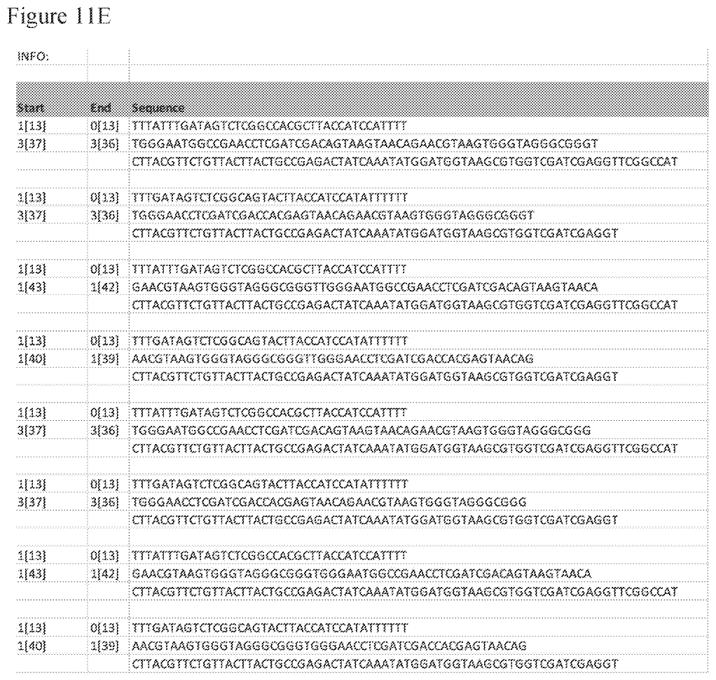

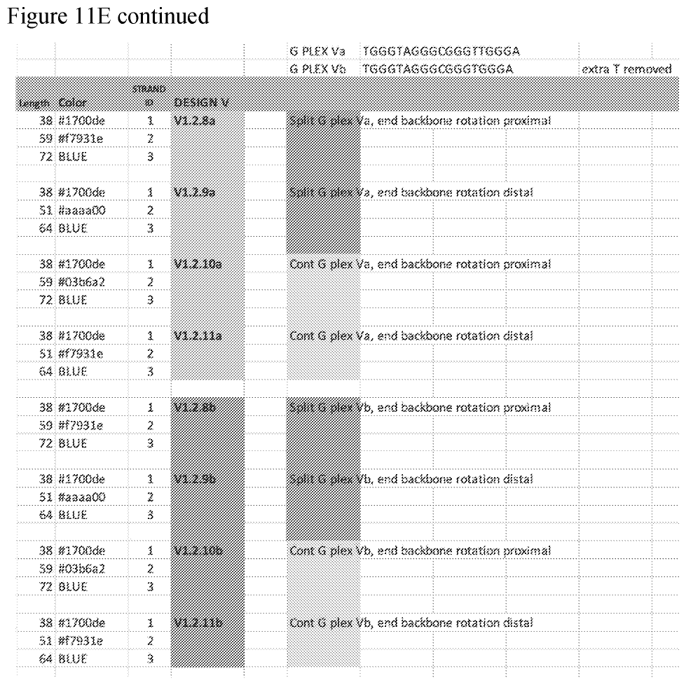

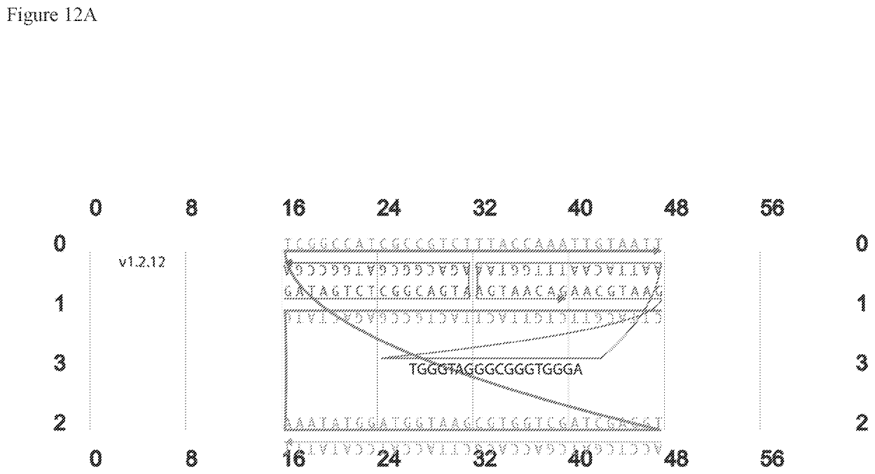

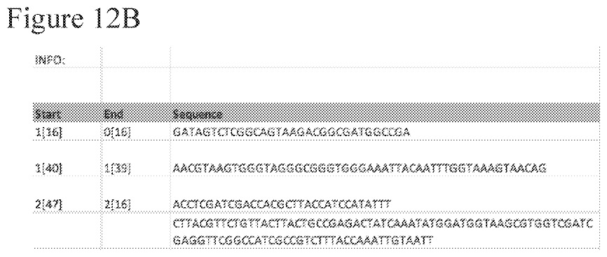

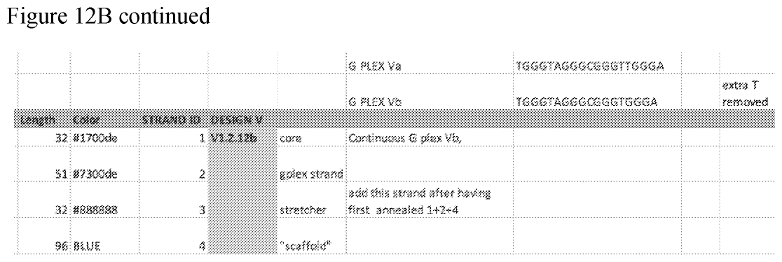

Constrained G plex (PDz) variants are depicted in FIGS. 11A-11E and include v1.2.8-v1.2.11. Each of these variants include two subvariants. v1.2.8a is an example of a split G plex variant with its end backbone rotated proximally, and v1.2.8b is an example of a split G plex variant with its end backbone rotated distally (FIG. 11A). v1.2.9a is an example of a split G plex variant with its end backbone rotated distally, and v1.2.9b is an example of a split G plex variant with its end backbone rotated proximally (FIG. 11B). v1.2.10a is an example of a continuous G plex variant with its end backbone rotated proximally, and v1.2.10b is an example of a continuous G plex variant with its end backbone rotated distally FIG. 11C). v1.2.11a is an example of a continuous G plex variant with its end backbone rotated distally, and v1.2.11b is an example of a continuous G plex variant with its end backbone rotated proximally (FIG. 11D). v1.2.12 G plex (PDz) is an exemplary variant inhibited by stretching (FIGS. 12A-12B).

The test design creates a configuration with the termini of the G plex being held about 10 nm apart. Testing of the activity is performed using ordered strand combinations. For example, the strand combinations may be strand 2 (SEQ ID NO 35); strand 2 (SEQ ID NO 35) then strand 3 (SEQ ID NO 33); strand 2 (SEQ ID NO 35), then strand 3 (SEQ ID NO 33), and then strand 1 (SEQ ID NO 34); strand 2 (SEQ ID NO 35), then strand 3 (SEQ ID NO 33), then strand 1 (SEQ ID NO 34), and then strand 3 (SEQ ID NO 32); or strand 2 (SEQ ID NO 35), then strand 3 (SEQ ID NO 33), and then 3 (FIG. 12B). These combinations are annealed together in order. Another annealing combination includes strand 1 (SEQ ID NO 34), strand 2 (SEQ ID NO 35), and then strand 3 (SEQ ID NO 33), and later annealed to strand 3 (SEQ ID NO 32) (FIG. 12B). Another annealing combination includes strand 2 (SEQ ID NO 35), and strand 3 (SEQ ID NO 32), and strand 3 (SEQ ID NO 33) later annealed to strand 1 (SEQ ID NO 34) (FIG. 12B).

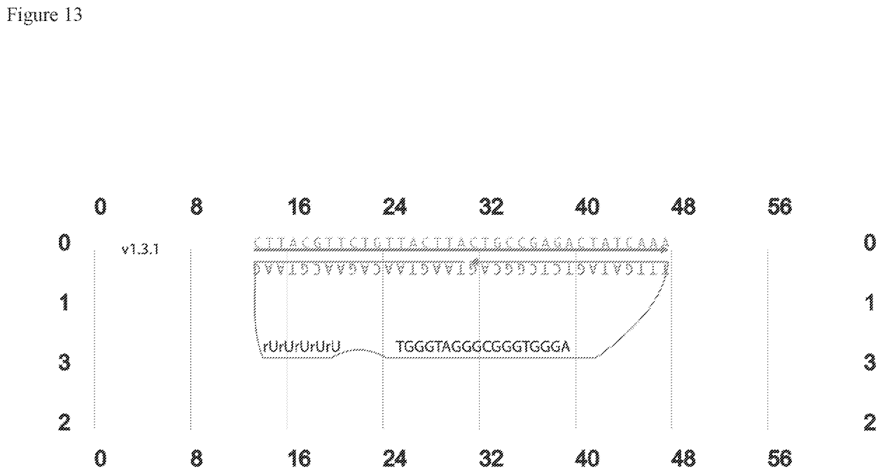

v1.3.1 and v1.3.2 are exemplary simplified G plex (PDz) designs which are inhibited by stretching and include a poly-U site for Cas cutting (FIGS. 13, 14A-14C). The test designs create a configuration with the termini of the G plex held about 13 nm apart. The nick site of v1.3.1 may allow for buckling, relieving mechanical stress, and may have a propensity to aggregate. v1.3.2 uses three strands to compensate for the potential nick-induced buckling of design v1.3.1. v1.3.2 may also form easily.

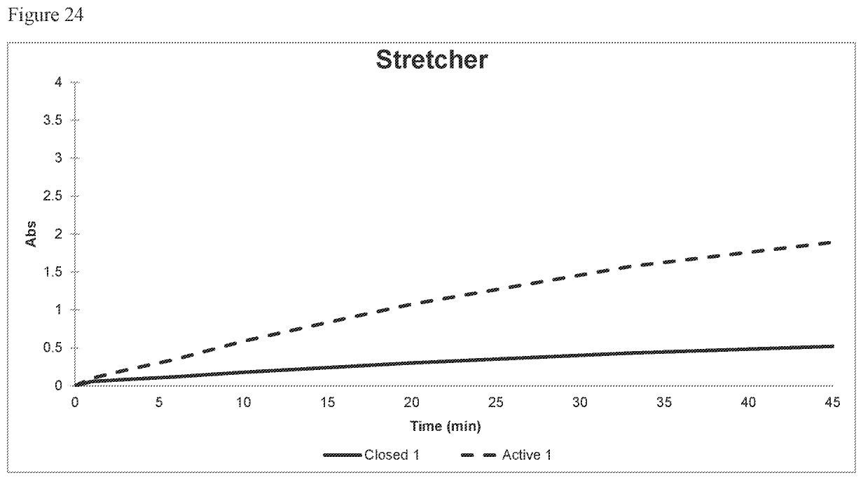

FIG. 24 depicts exemplary colorimetric data obtained stretched G4 quadruplex variants (v1.3.1 and v1.3.2). The dashed line represents fully active variants, and the solid line represents closed, inactive variants. The data provides absorbance measured by spectrophotometry.

Closed variants were formed by heating non-G4 strands between 500 and 80° C. for 5 to 20 minutes to anneal them together, cooling to room temperature with or without submerging in an ice bath for 10 to 90 minutes, adding the G4 strand before heating between 300 and 50° C. for 5 to 30 minutes, and finally cooling to room temperature with or without submerging in an ice bath for 10 to 90 minutes. Open variants contain an unstretched system, indicative of the system after the stretched strand is released by Cas in the amplifier output.

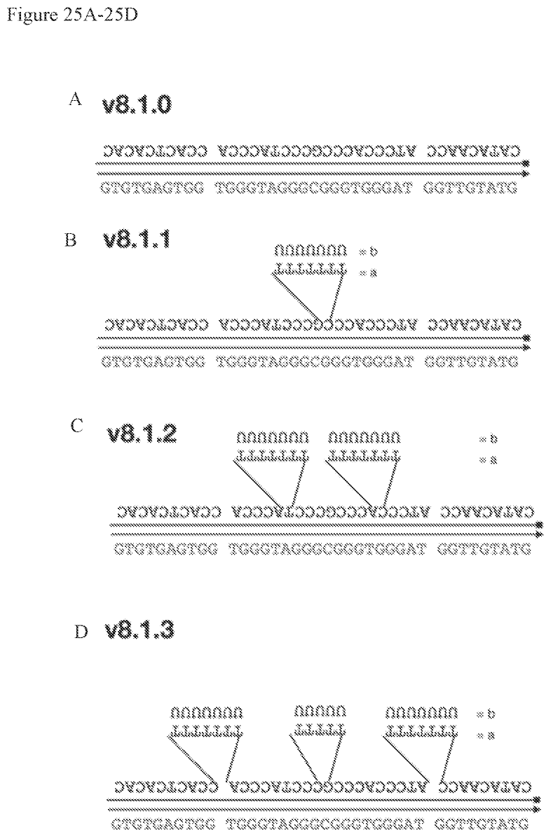

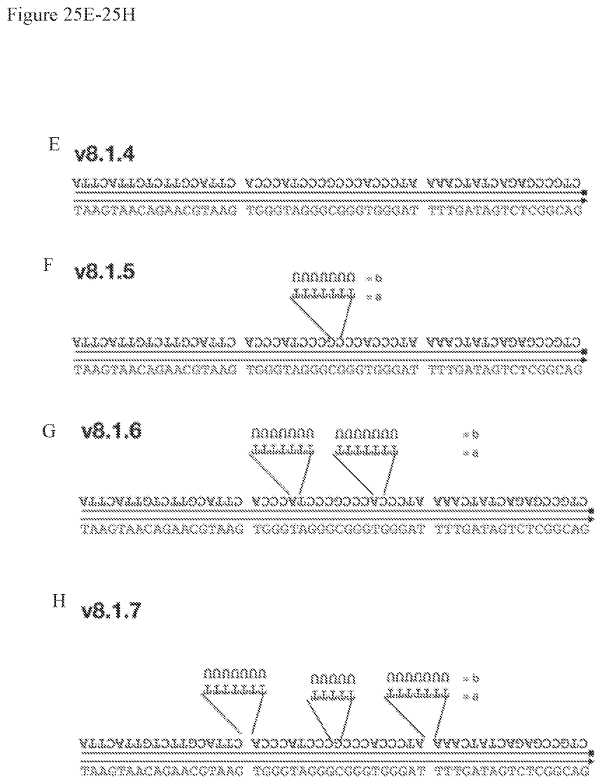

v8.1.0-v8.1.7 are examples of G plex caged duplexes (FIGS. 25A-25H). These designs are similar to G plex caged hairpin designs (FIGS. 8A-8E), however the system is composed of two strands. These variants may incorporate the reversible silencing of a G quadruplex peroxidase DNA molecule (G-plex strand) with a separate DNA molecule (silencing strand). To this end, the molecule containing the G quadruplex sequence motif is hybridized to a DNA oligonucleotide with complementary sequence. In addition to the G quadruplex sequence (TGGGTAGGGCGGGTGGGA (SEQ ID NO: 1)), there may be additional flanking sequence stretches that are used to increase the thermal stability of the duplex formed between both molecules. The silencing strand also may contain several poly-U loops in the region of the duplex with the G plex sequence motif. These poly U loops can be digested by a Cas13 nuclease. Such produced strand fragments of the silencing strand are able to independently dissociate from the G plex strand, which in turn can then fold up into a functional G quadruplex structure that can act as a peroxidase.

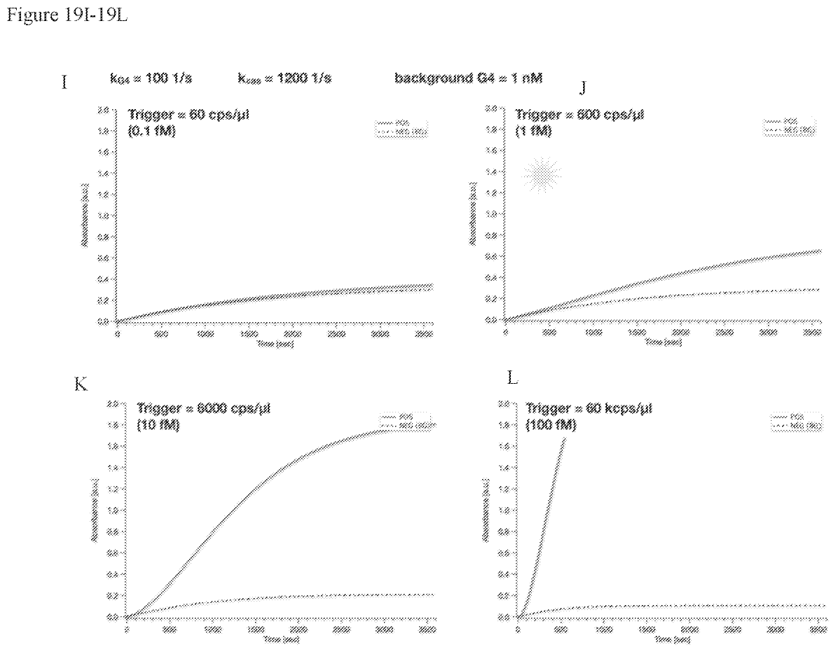

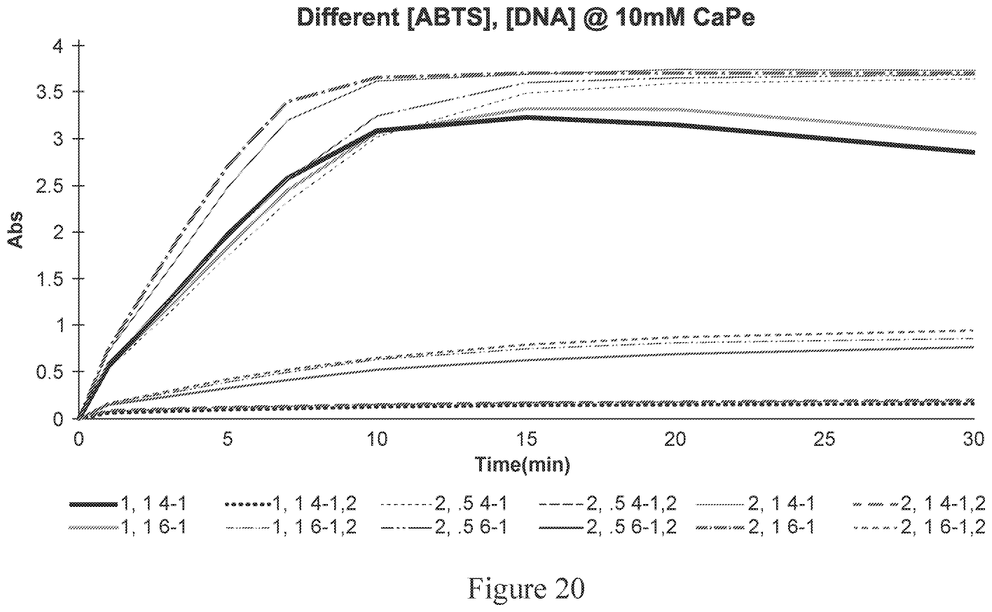

FIG. 20 depicts exemplary colorimetric data obtained with G4 quadruplex variants (v8.1.0-v8.1.7). The dashed lines represent fully active variants, and the solid lines represent closed, inactive variants. Shading pair the “on” and “off” states of the same variant preparation. Different shades correspond to different design variants. The absorbance was measured by spectrophotometry, where an absorbance value of 4 (arbitrary units) is represented by a thin line (e.g., lines 2, 14-1 and 2, 14-1, 2) and an absorbance value of 0.5 (arbitrary units) is represented by a thick line (e.g., lines 1, 14-1 and 1, 14-1, 2). The residual activity of closed variants is attributed to ill-formed hairpins that can be removed by, for example, further purifying for refining production of strands.

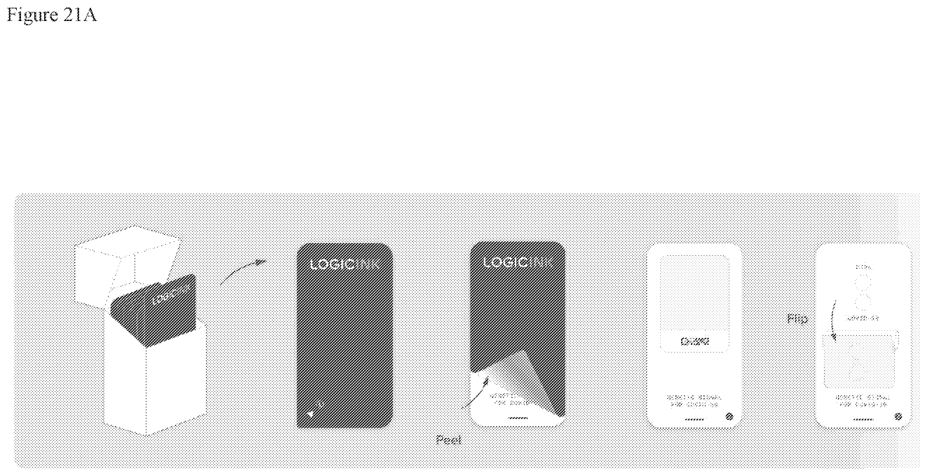

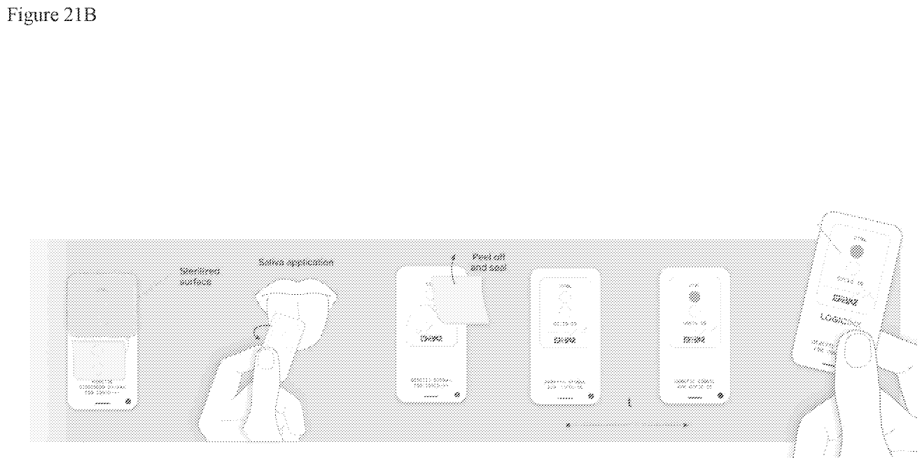

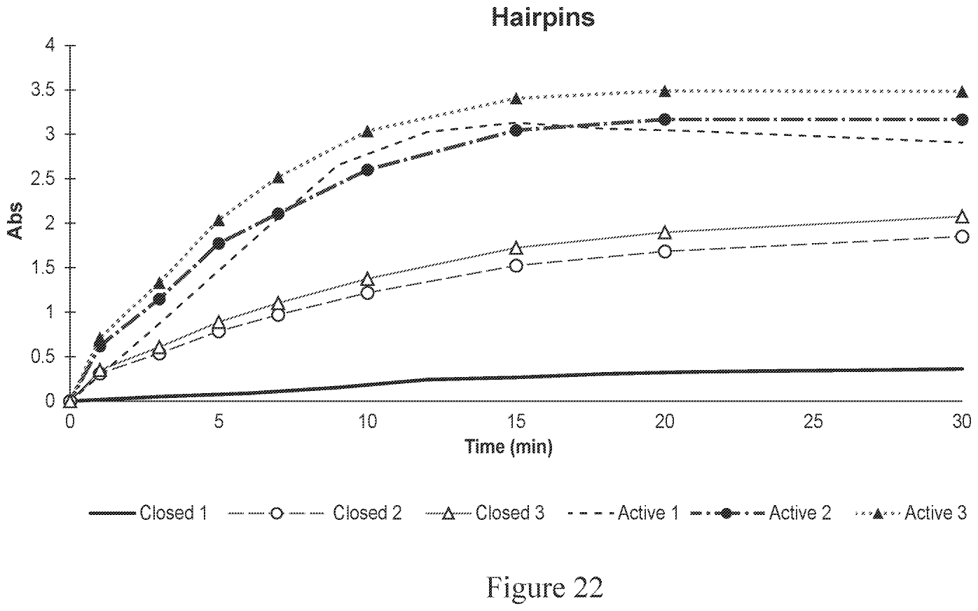

Closed variants were formed by heating between 700 and 95° C. for 5 to 20 minutes to melt the G4 structure, keeping them above 50° C. for 10 to 40 minutes, and cooling to room temperature with or without submerging in an ice bath for 10 to 90 minutes. Open variants contain two strands that will result after the hairpin loop is cleaved by Cas protein in the amplifier output. For example, FIG. 22 depicts G4 quadruplex variants (solid lines) and fully active variants (dashed lines).

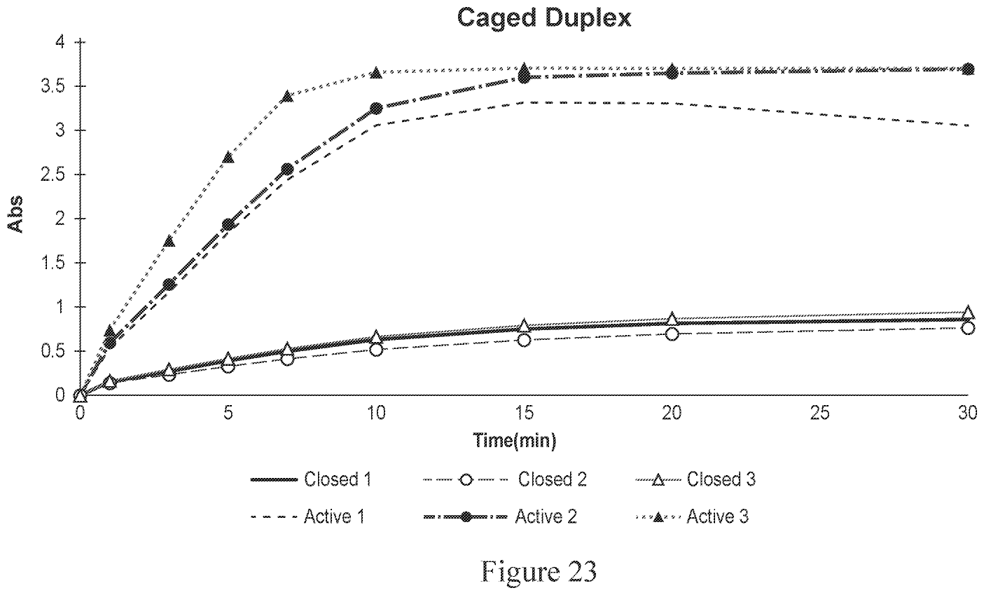

FIG. 23 depicts exemplary colorimetric data obtained with caged G4 quadruplex variants (v8.1.0-v8.1.7). The dashed lines represent fully active variants, and the solid lines represent closed, inactive variants. Shading pair the “on” and “off” states of the same variant preparation.

Different shades correspond to different design variants. The absorbance was measured by spectrophotometry, where an absorbance value of 4 (arbitrary units) is represented by a thin line (e.g., lines 2, 14-1 and 2, 14-1, 2) and an absorbance value of 0.5 (arbitrary units) is represented by a thick line (e.g., lines 1, 14-1 and 1, 14-1, 2).

Closed variants were formed by heating between 70° and 95° C. for 5 to 20 minutes to melt the G4 structure and cooling to room temperature with or without submerging in an ice bath for 10 to 90 minutes. Open variants contain the G4 strand that will be displaced after the complementary strand is cleaved/destroyed by Cas protein in the amplifier output.

### Example 4

### Exponential Increase of Indiscriminate Nuclease Activity

Provided here is the performance of exemplary systems that utilizes a sequence-programmable CRISPR-Cas13 collateral nuclease activity.

To exploit the collateral activity, a Cas13 protein is mixed with appropriately designed CRISPR RNA molecule (“crRNA”) to form a ribonucleoprotein complex. A target ssRNA with sequence that is complementary to a specific stretch on the crRNA can hybridize to the crRNA. As a result, Cas13 is activated and begins to indiscriminately digest ssRNA substrate molecules, irrespective of their sequence.

A Cas13-cleavable caged G-quadruplex hairpin construct is provided in the system. When Cas13 cuts the hairpin loop, the hairpin stem dissociates. One of the hairpin stem strands contains a G quadruplex sequence which can then fold into the G quadruplex and assumes peroxidase activity which can be used to generate a naked-eye readable colorimetric output. This is two-tiered enzyme cascade without feedback, in which target RNA activate Cas13 enzymes, which in turn catalyze the activation of peroxidases.

The cascade system can be modified to include a feedback loop, which will generate exponential amplification. In this manner, the exemplary system is autocatalytic. To this end, a crRNA-Cas13 complex is that responds to a target sequence (e.g. a SARS-Cov2 genomic RNA sequence motif) is used (FIG. 27A).

A crRNA-Cas13 complex that utilizes a Cascade-crRNA featuring the RNA version of the G-quadruplex sequence motif is provided (FIG. 27B). Once a SARS-Cov2 target RNA is detected by the corresponding Cas13-crRNA complex (FIG. 27A), the collateral activity of Cas13 as seeded by a viral target RNA leads to cleavage of the G-quadruplex hairpin motif that is also included in solution (FIGS. 27D-27E). The cut strands dissociate from one another. The G quadruplex strand folds and become an active peroxidase (FIG. 27F). The complementary strand (“Motiv 1”) (FIG. 27F) can now hybridize to the cascade crRNA1 of the corresponding Cas13 complex (FIG. 27B), leading to another Cas13 enzyme with collateral activity that is capable of digesting G quadruplex hairpin constructs (FIG. 27D). This results in an autocatalytic, self-amplifying exponential process that rapidly increases the amount of liberated G quadruplex strands.

Alternatively, a crRNA-Cas13 complex can be prepared that utilizes a cascade-crRNA that responds to a target RNA motif (e.g., another sequence) that can be, but does not need to be, fully independent of the viral target sequence (FIG. 27C). In addition to this crRNA-Cas13 complex, the system also provides another hairpin probe with a loop-stem structure as shown in FIG. 27H. Digestion of this hairpin loop by collateral activity of Cas13 as seeded by a viral target RNA leads to strand dissociation where one of the resulting strands able to hybridize to the cascade-crRNA2-Cas13 complex in FIG. 27C, which will lead to activation. Additional active Cas13 are thus generated that can cut both the amplifying hairpin probes (FIG. 27H) and also the G quadruplex hairpin probes (FIG. 27D). Exponential self-amplification will strongly enhance the signal. This exemplary system will not present background peroxidase activity. The second target RNA motif (e.g., another sequence) (FIG. 27H) may be the same sequence used to detect the initial SARS-Cov2 target in the Cas13 complex (FIG. 27A).

Whereas an initial trigger RNA viral motif may have extremely low concentration (e.g., about 1 fmol/L or less), the amplifying hairpin probes (FIGS. 27D and 27H) may be provided in high concentrations (e.g., about 500 nmol/L). This means the system is provided with a large pool of secondary trigger molecules for Cas13 that will provide strong exponential amplification as soon as the cascade is triggered.

Exemplary sequences for the cascade constructs include:

Cascade crRNA 1:
(SEQ ID NO: 2)
GACCACCCCAAAAAUGAAGGGGACUUGGGUAGGGCGGGUGGGA;
Cascade crRNA 2:
(SEQ ID NO: 3)
GACCACCCCAAAAAUGAAGGGGACUAAAACUUUCGCUGAUUUUGGGGU
CC;
G quadruplex hairpin:
(SEQ ID NO: 4)
UUUUUUCCCACCCGCCCUACCCAUUUUUUUUUUTGGGTAGGGCGGGGG
GA;
Cascade hairpin:
(SEQ ID NO: 5)
UUUUUGGACCCCAAAAUCAGCGAAAGUUUUUUUUUUAAAACUUUCGCU
GAUUUUGGGGUCC.

These systems may be combined with one another to enhance sensitivity.

### Example 5

### Use Cases

Provided here is a non-exhaustive list of use cases and embodiments of the systems and methods.

### Self-Testing

The user has a set of small thin cards at home. The user (1) takes out a card, (2) removes its cover, (3) applies saliva onto the microreservoirs on the card, (4) closes a lid covering the reservoirs, and (5) waits to learn if she's positive or negative (FIG. 15). The microreservoirs may be empty before saliva is deposited on it or may comprise a gel-like material necessary for extraction, inactivation, or sensing. The sensors may be printed on the lid, protruding from its interior face. Thus, when the lid is closed, the sensors are in contact with the saliva, allowing the user to potentially lick the reservoirs knowing that there would be no contact with the sensor. A negative result will show the control sensor turn color only. A positive result will result in 2 or 3 or more sensors turning color.

Self Testing with Voluntary Mobile App Reporting

Similar to self-testing, but a computer vision mobile app scans the card and sends the results to a central location (e.g., for contact tracing).

Self-Testing with Compulsory Mobile App Reporting

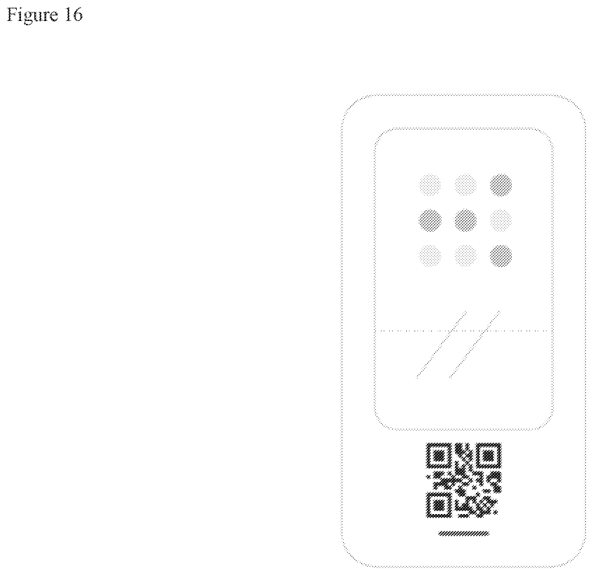

Similar to self-testing with voluntary mobile app reporting. However, the meaning of each sensor is unknown to the user. Additional sensors and corresponding reservoirs may be added where the sensors may only need saliva to be activated (e.g., similar to an electrolyte sensor or a pH sensor). In some cases, there may be no sensor thus the material in contact with the saliva would never change color. Additionally, the card may have a printed key using a barcode or QR code, or some other way that can be read by the mobile app (FIG. 16). The test results and key are read by the mobile app and sent to a central location. The central location uses the key to determine the meaning of all the reservoirs and sends back the test results to the mobile app user. Even though the number of possible reservoir configurations is limited, the key can be unique to each card. Thus, knowledge of results of one card does not determine knowledge of the configuration of other cards.

### Supervised Test

Similar to the above uses, but the test is done in the presence of someone approved to validate the test. For example, when entering a stadium, airline, school, restaurants, work, etc., a test may be done while being supervised by someone at the airline, school, stadium, etc. tasked to ensure that the test was correctly performed (FIG. 17). This supervised test may be in addition to a self-test performed by the user earlier that day at home our just outside the venue being attended. Additionally, the job of the person tasked to ensure the test is correctly performed may include verifying that this person is who she says she is.

In all uses discussed above, the card may be placed on the user's hand or some visible place and act as a health pass.

As will be apparent to one of ordinary skill in the art from a reading of this disclosure, the present disclosure can be embodied in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein.

All references and publications recited are incorporated by reference.

1. A system comprising:
a translation module comprising:
a first nucleic acid comprising a first polynucleotide, and
a second nucleic acid comprising a second polynucleotide, wherein the second polynucleotide is configured to reversibly hybridize the first polynucleotide; and
wherein the translation module is configured to accept one or more input signals,
wherein the one or more input signals comprise one or more target nucleic acids,
wherein the one or more target nucleic acids comprise a target polynucleotide,
wherein the first polynucleotide is configured to hybridize the target polynucleotide, and
wherein the second polynucleotide is configured to dissociate from the first polynucleotide to provide a translator output;
an amplification module comprising one or more sensing modalities,
wherein the amplification module is configured to accept the translator output,
wherein the one or more sensing modalities are configured to detect the translator output, and
wherein the one or more sensing modalities are configured to provide an amplifier output upon detecting the translator output; and
a detection module configured to accept the amplifier output,
wherein the detection module comprises one or more substrates and one or more activatable nucleic acid peroxidases,
wherein the one or more activatable nucleic acid peroxidases are configured to be converted into one or more active nucleic acid peroxidases in the presence of the amplifier output, and
wherein the one or more active nucleic acid peroxidases are configured to convert the one or more substrates into one or more products.

2. The system of claim 1, wherein the one or more sensing modalities comprises one or more of:
one or more isothermal chemical ligation-hybridization and chemical cross replication modules configured to replicate the translator output as the amplifier output,
one or more hairpin-chain reaction modules configured to replicate the translator output as the amplifier output,
one or more duplicator gate cascade modules configured to replicate the translator output as the amplifier output,
one or more CRISPR-Cas13 modules configured to provide an active Cas13 complex as the amplifier output,
one or more CRISPR-Cas12 modules configured to provide an active a Cas12 complex as the amplifier output, or
one or more nucleated polymerization module configured to provide a polymer as the amplifier output.

3. The system of any of claims 1 to 2, wherein the one or more activatable nucleic acid peroxidases are one or more of:
one or more caged nucleic acid peroxidases comprising a digestible region, wherein the amplifier output is configured to digest the digestible region, and wherein the one or more caged nucleic acid peroxidases are configured to be converted to the one or more active nucleic acid peroxidases upon digestion of the digestible region,
one or more nucleic acid peroxidases in tension, wherein the amplifier output is configured to relax the one or more nucleic acid peroxidases in tension, and wherein the one or more nucleic acid peroxidases are configured to be converted to the one or more active nucleic acid peroxidases upon relaxation, or
one or more first peroxidase polynucleotides, and one or more second peroxidase polynucleotides, wherein the one or more first peroxidase polynucleotide and the one or more second peroxidase polynucleotide are configured to form the one or more active nucleic acid peroxidases when the one or more first peroxidase polynucleotide are in proximity to the one or more second peroxidase polynucleotide, wherein the one or more activatable nucleic acid peroxidases are configured to bring the one or more first peroxidase polynucleotides and the one or more second peroxidase nucleotides into proximity when the amplifier output is present.

4. The system of claim 1, wherein the one or more sensing modalities comprises one or more of one or more CRISPR-Cas13 modules configured to provide an active Cas13 complex as the amplifier output or one or more CRISPR-Cas12 modules configured to provide an active Cas12 complex as the amplifier output;
wherein the one or more activatable nucleic acid peroxidases are one or more first peroxidase polynucleotides, and one or more second peroxidase polynucleotides, wherein the one or more first peroxidase polynucleotide and the one or more second peroxidase polynucleotide are configured to form the one or more active nucleic acid peroxidases when the one or more first peroxidase polynucleotide are in proximity to the one or more second peroxidase polynucleotide; and
wherein the detection module is configured to bring the one or more first peroxidase polynucleotides and the one or more second peroxidase nucleotides into proximity when the amplifier output is present.

5. The system of claim 4, further comprising one or more reporter nucleic acids,
wherein the one or more reporter nucleic acids are configured to hold the one or more first peroxidase polynucleotides separate from the one or more second peroxidase polynucleotides when the one or more reporter nucleic acid is not cleaved,
wherein the one or more activatable nucleic acid peroxidases are configured to bring the one or more first peroxidase polynucleotides into proximity with the one or more second peroxidase polynucleotides when the one or more reporter nucleic acids is cleaved, and
wherein the amplifier output is configured to cleave the one or more reporter nucleic acids.

6. The system of claim 5, wherein the system comprises a plurality of activatable nucleic acid peroxidases, wherein each activatable nucleic acid peroxidase comprises one of the one or more first peroxidase polynucleotide and one of the one or more second peroxidase polynucleotide, and wherein the detection module comprises:
a first portion comprising a first surface, the one or more first peroxidase polynucleotides attached to the first surface,
a second portion comprising a second surface, the one or more second peroxidase polynucleotides attached to the second surface, and
a hinge connecting the first portion and the second portion, wherein the hinge is configured to hold the detection module in an open form when the one or more reporter nucleic acids is uncleaved or the one or more reporter nucleic acids is single stranded, and wherein the hinge is configured to hold the detection module in a closed form when the one or more reporter nucleic acids is cleaved or the one or more reporter nucleic acids is double stranded, and
wherein the one or more first surface and the second surface are configured to separate the one or more first peroxidase polynucleotides and the one or more second peroxidase polynucleotides when the detection module is in the open form, and wherein the first surface and the second surface are configured to bring the one or more first peroxidase polynucleotides into proximity with the one or more second peroxidase polynucleotides when the detection module is in the closed form.

7. The system of claim 1, wherein the one or more sensing modalities comprises one or more of one or more CRISPR-Cas13 modules configured to provide an active Cas13 complex as the amplifier output or one or more CRISPR-Cas12 modules configured to provide an active a Cas12 complex as the amplifier output; and
wherein the one or more activatable nucleic acid peroxidases are one or more nucleic acid peroxidases in tension, wherein the amplifier output is configured to relax the one or more nucleic acid peroxidases in tension, and wherein the one or more nucleic acid peroxidases are configured to be converted to the one or more active nucleic acid peroxidases upon relaxation.

8. The system of claim 7, further comprising one or more reporter nucleic acids,
wherein the one or more reporter nucleic acids are configured to hold the one or more nucleic acid peroxidases in tension when the one or more reporter nucleic acid is not cleaved, and
wherein the amplifier output is configured to cleave the one or more reporter nucleic acids.

9. The system of claim 8, wherein the system comprises a plurality of activatable nucleic acid peroxidases, and wherein the detection module comprises:
a first portion comprising a first surface, wherein the one or more nucleic acid peroxidases are attached to the first surface,
a second portion comprising a second surface, the wherein the one or more nucleic acid peroxidases are attached to the second surface, and
a hinge connecting the first portion and the second portion, wherein the hinge is configured to hold the detection module in an open form when the one or more reporter nucleic acids is uncleaved, and wherein the hinge is configured to hold the detection module in a closed form when the one or more reporter nucleic acids is cleaved, and
wherein the one or more first surface and the second surface are configured to hold the one or more nucleic acid peroxidases in tension when the detection module is in the open form, and wherein the first surface and the second surface are configured to hold the one or more nucleic acid peroxidases in tension when the detection module is in the closed form.

10. The system of any of the proceeding claims,
wherein the amplification module further comprises one or more activatable elements, wherein the amplifier output is configured to activate the one or more activatable elements to provide one or more activated elements, wherein the one or more activated elements are configured to detect one or more nucleic acids, and wherein the one or more sensing modalities are configured to provide a second amplifier output upon detecting the one or more nucleic acids; and
wherein the detection module is further configured to accept the second amplifier output, and wherein the one or more activatable nucleic acid peroxidases are configured to be converted into the one or more active nucleic acid peroxidases in the presence of the second amplifier output.

11. The system of claim 10, wherein the one or more activatable elements is a masked nucleic acid, and wherein the one or more activated elements is not masked.

12. The system of any of claims 10 and 11, wherein the second amplifier output and the amplifier output are the same.

13. The system of any of claims 10 to 12, wherein the one or more activated elements and the translator output are the same.

14. The system of any of the proceeding claims, wherein the one or more input signals is provided by dissociating the one or more target nucleic acids of the one or more input signals from another polynucleotide.

15. A method for detecting one or more target nucleic acids, wherein the one or more target nucleic acids comprises a target polynucleotide, the method comprising:
providing a sample to a translation module, the translation module comprising:
a first nucleic acid comprising a first polynucleotide, and
a second nucleic acid comprising a second polynucleotide, wherein the second polynucleotide is configured to reversibly hybridize the first polynucleotide;
hybridizing the first polynucleotide to the target polynucleotide;
dissociating the second polynucleotide from the first polynucleotide to provide a translator output;
providing the translator output to an amplification module, wherein the amplification module comprises one or more sensing modalities;
detecting the translator output by the one or more sensing modalities;
providing an amplifier output from the one or more sensing modalities;
providing the amplifier output to a detection module, wherein the detection module comprises one or more substrates and one or more activatable nucleic acid peroxidases;
converting the one or more activatable nucleic acid peroxidases into one or more active nucleic acid peroxidases; and
converting the one or more substrates into one or more products with the one or more active nucleic acid peroxidases.

16. The method of claim 15, wherein the step of providing the amplifier output from the one or more sensing modalities comprises one or more of:
replicating the translator output by isothermal chemical ligation-hybridization and chemical cross replication,
replicating the translator output by hairpin-chain reaction,
replicating the translator output with duplicator gate cascades,
providing an active Cas13 complex,
providing an active a Cas12 complex, or
providing a polymer by nucleated polymerization.

17. The method of any of claims 15 to 16, wherein the step of converting the one or more activatable nucleic acid peroxidases into one or more active nucleic acid peroxidases comprises one or more of:
digesting a digestible region of one or more caged nucleic acid peroxidases,
relaxing one or more nucleic acid peroxidases in tension, or
bringing one or more first peroxidase polynucleotides and one or more second peroxidase polynucleotides into proximity.

18. The method of claim 15, wherein the step of providing the amplifier output from the one or more sensing modalities comprises providing one or more of an active Cas13 complex and an active a Cas12 complex; and
wherein the step of converting the one or more activatable nucleic acid peroxidases into one or more active nucleic acid peroxidases comprises one or more of one or more first peroxidase polynucleotides and one or more second peroxidase polynucleotides into proximity.

19. The method of claim 18, wherein the step of converting the one or more activatable nucleic acid peroxidases into one or more active nucleic acid peroxidases comprises cleaving one or more reporter nucleic acids with the active Cas13 complex or active Cas12 complex, and
wherein the one or more reporter nucleic acids are configured to hold the one or more first peroxidase polynucleotides separate from the one or more second peroxidase polynucleotides when the one or more reporter nucleic acid is not cleaved.

20. The method of claim 15, wherein the step of providing the amplifier output from the one or more sensing modalities comprises providing one or more of an active Cas13 complex and an active a Cas12 complex; and
wherein the step of converting the one or more activatable nucleic acid peroxidases into one or more active nucleic acid peroxidases comprises relaxing one or more nucleic acid peroxidases in tension.

21. The method of claim 20, wherein the step of converting the one or more activatable nucleic acid peroxidases into one or more active nucleic acid peroxidases comprises cleaving one or more reporter nucleic acids with the active Cas13 complex or active Cas12 complex, and
wherein the one or more reporter nucleic acids are configured to hold the one or more activatable nucleic acid peroxidases in tension when the one or more reporter nucleic acid is not cleaved.

22. The method of any of claims 15 to 21, wherein the amplification module further comprises one or more activatable elements, and wherein the method further comprises:
activating the one or more activatable elements with the amplifier output,
providing one or more activated elements,
detecting one or more nucleic acids with the one or more activated elements,
providing a second amplifier output, and
providing the second amplifier output to the detection module.

23. The system of claim 22, wherein the one or more activatable elements is a masked nucleic acid, and wherein the one or more activated elements is not masked.

24. The system of any of claims 22 and 23, wherein the second amplifier output and the amplifier output are the same.

25. The system of any of claims 22 to 24, wherein the one or more activated elements and the translator output are the same.

26. The system of any of the proceeding claims further comprising dissociating the one or more target nucleic acids of the one or more input signals from another polynucleotide.
